(19) United States
(12) Patent Application Publication
(10) Pub. No.: US 2023/0303712 A1
(43) Pub. Date: Sep. 28, 2023

(54) METHODS OF TREATING GLIOBLASTOMA

(71) Applicant: Ramot at Tel-Aviv University Ltd., Tel-Aviv (IL)

(72) Inventors: Ronit SATCHI-FAINARO, Tel-Aviv (IL); Eilam YEINI, Tel-Aviv (IL)

(73) Assignee: Ramot at Tel-Aviv University Ltd., Tel-Aviv (IL)

(21) Appl. No.: 18/122,194

(22) Filed: Mar. 16, 2023

Related U.S. Application Data
(63) Continuation of application No. PCT/IL2021/051126, filed on Sep. 14, 2021.
(60) Provisional application No. 63/135,783, filed on Jan. 11, 2021.
(60) Provisional application No. 63/079,008, filed on Sep. 16, 2020.

(51) Int. Cl. C07K 16/28; A61K 31/554; C12N 15/113; A61P 35/00; A61P 25/00; A61K 45/06

(52) U.S. Cl. CPC C07K 16/2896; A61K 31/554; A61K 45/06; A61P 25/00; A61P 35/00; C12N 15/1138; C07K 2317/76; C12N 2310/531

(57) ABSTRACT

A method of treating glioblastoma in a subject in need thereof is disclosed. The method comprises administering to the subject a therapeutically effective amount of an agent that specifically decreases an amount and/or activity of P-selectin.

### RELATED APPLICATIONS

This application is a Continuation of PCT Application No. PCT/IL2021/051126 which claims the benefit of priority under 35 USC § 119(e) of US Pat. Application Nos. 63/079,008 filed on Sep. 16, 2020, and 63/135,783 filed on Jan. 11, 2021. The contents of the above applications are all incorporated by reference as if fully set forth herein in their entirety.

### SEQUENCE LISTING STATEMENT

The XML file, entitled 94997Sequence Listing.xml, created on Mar. 16, 2023, comprising 23,807 bytes, submitted concurrently with the filing of this application is incorporated herein by reference.

### FIELD AND BACKGROUND OF THE INVENTION

The present invention, in some embodiments thereof, relates to a method of treating glioblastoma by selectively decreasing the activity or amount of P-selectin.

Glioblastoma (GB), the most common and lethal type of primary brain tumor, is a highly heterogeneous tumor characterized by enhanced angiogenesis. Even with the latest standard of care, which includes surgery followed by chemotherapy and radiotherapy, the median survival is only 20 months. Complete surgical removal of the tumor is challenging due to the invasive nature of the disease. Therefore, uncovering new therapeutic targets involved in GB establishment and progression could be of immense use. It is well known that the tumor microenvironment and the immune system play an important role in tumor progression. Although T cells are not abundant in the brain microenvironment, the myeloid lineage comprises 30% of the cells found in GB tumors, with recent accumulating evidence for their involvement in GB tumorigenesis.

Microglia are macrophage-like cells that serve as the brain immune system, and like macrophages, they can be found in at least two different activation states. A common classification categorizes microglia as M1 and M2 activation states where M1 is considered to represent the classical, pro-inflammatory state in which microglia are phagocytotic, cytotoxic, and possess antigen presentation capabilities. This classical activation allows microglia to attack transformed-cancerous cells and harness cytotoxic T cells against the tumor. In contrast, M2-like activation is considered to be the alternative, immune-suppressive state, which is related to tissue repair. This state is characterized by tissue remodeling and angiogenesis properties and the secretion of anti-inflammatory cytokines such as IL-10 and TGF-β.

Although mixed populations are found in GB tumors, which included both M1 and M2 subtypes, glioma-associated microglia/macrophages (GAMs) contribute to immune-escape and promote tumor progression, and have been shown to actively enhance glioma growth and invasion, and secrete angiogenic factors. Despite the evidence for the role of GAM in gliomas, current immunotherapies have not yet been demonstrated to improve survival for GB patients in a clinically significant manner. This emphasizes the need for alternative or combination approaches. Although the exact role and activation state of GAMs are still unclear, reverting their phenotype to an M1-like state has been shown to be a promising therapeutic approach for glioma and might also improve the outcome of immunotherapies in this disease. Several targets associated with this phenotypic switch, such as CSF-1R and STAT3, are currently under pre-clinical and clinical investigation for the treatment of GB [21-23].

Hence, uncovering new immune-modulators that could regulate microglia phenotype in GB is of great importance and could lead to the development of new therapeutic strategies to treat this devastating disease.

Background art includes US Pat. Application No. 20200171064 (which teaches isoquercetin or quercetin for treatment of cancer, including glioblastoma) and U.S. Pat. Application No. 20190241665 (which teaches P-selectin inhibitors for the treatment of metastasized cancers).

Shamay et al., Science Translational Medicine, Volume 8, issue 345, 29 June, 2016 teaches that P-selectin is expressed on cancer cells in many human tumors.

Ferber et al., eLife 2017;6:e25281. DOI: www(dot)doi(dot)org/10(dot)7554/eLife(dot)252 81 teaches that P-selectin is not only expressed on tumor endothelium but also on glioblastoma cells and may be used as a target for selective delivery of anti-cancer agents.

### SUMMARY OF THE INVENTION

According to an aspect of the present invention there is provided a method of treating glioblastoma in a subject in need thereof comprising administering to the subject a therapeutically effective amount of an agent that specifically decreases an amount and/or activity of P-selectin, thereby treating the glioblastoma.

According to an aspect of the present invention there is provided a pharmaceutical composition comprising a pharmaceutically acceptable carrier and:
(i) an active agent that specifically decreases an amount and/or activity of P-selectin; and
(ii) an immunomodulatory agent, as a second active agent.

According to an aspect of the present invention there is provided an article of manufacture comprising:
(i) an active agent that specifically decreases an amount and/or activity of P-selectin; and
(ii) an immunomodulatory agent.

According to embodiments of the present invention, the agent specifically binds to P-selectin or a polynucleotide encoding the P-selectin.

According to embodiments of the present invention, the agent binds to P-Selectin glycoprotein ligand-1 (PSGL-1) or a polynucleotide encoding the PSGL-1.

According to embodiments of the present invention, the method further comprises administering to the subject an immunomodulatory agent.

According to embodiments of the present invention, the immunomodulatory agent comprises an immunomodulatory antibody.

According to embodiments of the present invention, the immunomodulatory antibody is selected from the group consisting of anti-CTLA4, anti-CD40, anti-41BB, anti-OX40, anti-PD1, anti-PDL1, anti-LAG3, anti-IDO, and anti-TIGIT.

According to embodiments of the present invention, the agent is an inhibitory antibody that binds to and inhibits the P-selectin.

According to embodiments of the present invention, the inhibitory antibody is attached to a therapeutic agent.

According to embodiments of the present invention, the inhibitory antibody is not attached to a therapeutic agent.

According to embodiments of the present invention, the agent is a small molecule agent.

According to embodiments of the present invention, the agent is a polynucleotide agent.

According to embodiments of the present invention, the agent is co-formulated with the immunomodulatory agent.

According to embodiments of the present invention, the agent is administered following resection of the glioblastoma tumor.

According to embodiments of the present invention, the glioblastoma is an early stage glioblastoma.

According to embodiments of the present invention, the agent is comprised in a nanoparticle.

According to embodiments of the present invention, the nanoparticle is attached to a targeting moiety that increases delivery across the blood brain barrier.

According to embodiments of the present invention, the agent is attached to a targeting moiety that increases delivery across the blood brain barrier.

According to embodiments of the present invention, when the agent is an inhibitory antibody, the agent is not attached to a therapeutic agent.

According to embodiments of the present invention, the active agent specifically binds to P-selectin or a polynucleotide encoding the P-selectin.

According to embodiments of the present invention, the active agent binds to P-Selectin glycoprotein ligand-1 (PSGL-1) or a polynucleotide encoding the PSGL-1.

According to embodiments of the present invention, the immunomodulatory agent comprises an immunomodulatory antibody.

According to embodiments of the present invention, the immunomodulatory antibody is selected from the group consisting of anti-CTLA4, anti-CD40, anti-41BB, anti-OX40, anti-PD1, anti-PDL1, anti-LAG3, anti-IDO and anti-TIGIT.

According to embodiments of the present invention, the active agent is an inhibitory antibody that binds to and inhibits the P-selectin.

According to embodiments of the present invention, the active agent is a small molecule agent.

According to embodiments of the present invention, the active agent is a polynucleotide agent.

According to embodiments of the present invention, the active agent and the immunomodulatory agent are comprised in a nanoparticle.

According to embodiments of the present invention, the nanoparticle is attached to a targeting moiety that increases delivery across the blood brain barrier.

According to embodiments of the present invention, the active agent is attached to a targeting moiety that increases delivery across the blood brain barrier.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A, 1B, 1C, 1D, 1E, 1F, 1G, 1H, 1I and 1J. Microglia facilitates the proliferation and migration rate of GB cells and enhances the expression of SELP by GB cells. A. Iba1 immunostaining showing activated microglia in GB tumors compared to normal/adjacent tissue in a GB patient FFPE sample and three GB mouse models; iRFP-labeled human U251, patient-derived (PD-GB4) GB xenografts and mCherry-labeled murine GL261, N = 3 mice or 3 patient samples. Scale bars represent 100 µm. B-C. The proliferation (B) and migration (C) rates of iRFP-PD-GB4 GB cells were enhanced in the presence of human microglia. Data represent mean ± s.d. of triplicate wells. The graphs are representative of three independent repeats. Statistical significance was determined using an unpaired, two-sided Student’s t-test. D. Cytokine profile showing over-secretion of SELP and other factors in a co-culture of human PD-GB4 GB cells and primary human microglia compared to monocultures. Data represent mean + s.d. Duplicates were measured for each cytokine. The graph shows the average of two independent studies including internal repeats. E-F. ELISA (E) and Real-time PCR (F) results showing over secretion and mRNA expression of SELP by PD-GB4 cells when treated with human microglia CM (MG CM) compared to naive microglia medium. Data represent mean ± s.d. Each dot represents a triplicate. The graphs show the average of three independent studies. Statistical significance was determined using an unpaired, two-sided Student’s t-test. G-H. Representative image (G) and quantification (H) of flow cytometry analysis showing over-expression of SELP when PD-GB4 spheroids were treated with microglia CM compared to naive microglia medium. Data represent mean ± s.d. The graph shows the average of three independent studies. Statistical significance was determined using an unpaired, two-sided Student’s t-test. I. SELP and PSGL-1 are highly express in tumor areas enriched with activated microglia in GB mouse models. N = 3 mice per staining. Scale bars represent 100 µm. J. SELP is highly expressed in short-term survivors (STS) GB patient FFPE samples compared to long-term survivors (LTS) or normal human brain tissue. Data represent mean ± s.d. Each dot represents the average of three images per sample. N = 5 human samples per group. Scale bars represent 100 µm. Statistical significance was determined using one-way ANOVA test with multiple comparisons adjustment.

FIGS. 2A, 2B, 2C, 2D, 2E and 2F. SELP mediates GB cell invasion, migration, and proliferation. A. Real-time PCR showing reduced SELP mRNA levels in shSELP PD-GB4 cells, compared to control WT (Control), and negative control shRNA (shNC). Data represent mean ± s.d. Each dot represents a triplicate. The graph showing the average of three independent experiments. Statistical significance was determined using one-way ANOVA test with multiple comparisons adjustment. B-C. Representative image (B) and quantification (C) of Flow cytometry analysis of SELP expression showing reduced expression in shSELP PD-GB4 compared to control WT and shNC PD-GB4 cells. Data represent mean ± s.d. The graph shows the average of three independent experiments. Statistical significance was determined using one-way ANOVA test with multiple comparisons adjustment. D. Proliferation of iRFP-labeled PD-GB4 cells alone or co-cultured with unlabeled human microglia showing the lower proliferation rate of shSELP PD-GB4 compared to control WT and shNC PD-GB4 cells. Cell proliferation was followed and analyzed using the IncuCyte imaging system for 72 h. Data represent mean ± s.d. of triplicate wells. The graph a is representative of three independent repeats. Statistical significance was determined using two-ways ANOVA test with multiple comparisons adjustment. E. Wound healing assay using iRFP-labeled PD-GB4 cells co-cultured with microglia, showing the lower migration rate of shSELP PD-GB4 compared to control WT and shNC PD-GB4 cells. Wound closure was followed and analyzed by the IncuCyte imaging system for 60 h. Data represent mean ± s.d. of 4 wells per group. The graph is a representative of three independent repeats. Statistical significance was determined using two-ways ANOVA test with multiple comparisons adjustment. F. 3D spheroid invasion in Matrigel showing enhanced invasion of iRFP-labeled PD-GB4 GB cells when co-culture with unlabeled human microglia, and reduced invasion following SELP knockdown or treatment with 0.5 µM SELPi compared to untreated control WT or shNC PD-GB4 cells. Sprouting was followed for 72 h. Data represent mean ± s.d. of 5 spheroids per group. The graph is a representative of three independent experiments. Scale bars represent 100 µm. Statistical significance was determined using one-way ANOVA test with multiple comparisons adjustment.

FIGS. 3A, 3B, 3C, 3D, 3E, 3F, 3G, 3H, 3I, 3J and 3K. SELP inhibits the microglia pro-inflammatory phenotype and promotes their immunosuppressive activity. A. ELISA assay showing higher secretion of SELP by human microglia treated with PD-GB CM compared to naive DMEM. Data represent mean ± s.d. Each dot represents a triplicate. The graph shows the average of three independent repeats. Statistical significance was determined using an unpaired, two-sided Student’s t-test. B. Real-Time PCR showing higher expression SELP mRNA by human microglia treated with PD-GB4 CM compared to naive DMEM. Data represent mean ± s.d. Each dot represents a triplicate. The graph shows the average of three independent repeats. Statistical significance was determined using an unpaired, two-sided Student’s t-test. C-D. Representative image (C) and quantification (D) of flow cytometry analysis of PSGL-1 expression by human microglia, showing higher expression when treated with PD-GB4 GB CM compared to naive DMEM. Data represent mean ± s.d. The graph shows the average of five independent experiments. Statistical significance was determined using an unpaired, two-sided Student’s t-test. E. tSNE plot of single-cell RNA-seq analysis of microglia and macrophages isolated from patients’ GB tumors, showing the expression of PSGL-1 by microglia compared to macrophages. Data obtained from Darmanis et al. [56]. F-I. Treatment with rSELP resulted in elevated mRNA expression of ARG1 (F), reduced expression of iNOS (G) and elevated expression of IL-10 (H) and TGF-β (I) by human microglia. SELPi or anti-PSGL-1 neutralizing antibody rescued rSELP effects while anti-CD44 or CD24 neutralizing antibody did not affect gene expression. Data represent mean + s.d. Each dot represents a triplicate. The graphs show the average of three independent studies. Statistical significance was determined using one-way ANOVA test with multiple comparisons adjustment. J-K. Phagocytic activity (J) and NO release (K) by human microglia were reduced when treated with rSELP, and were restored when inhibiting SELP or PSGL-1 but not when neutralizing CD44 or CD24. Data represent mean ± s.d. of four wells per group, three fields per well. The graphs are representative of three independent repeats. Scale bars represent 200 µm. Statistical significance was determined using one-way ANOVA test with multiple comparisons adjustment.

FIGS. 4A, 4B, 4C, 4D, 4E and 4F. SELP-knockdown inhibits tumor growth and prolongs survival in human GB mouse models. A. SELP knockdown reduced tumor growth rate of PD-GB4 tumors in mice compared to control WT (control) or shNC GB tumors. Data represent mean ± s.e.m. N = 8 control, 9 shNC, and 14 shSELP. One-way ANOVA, Dunn’s method, p < 0.001 B. Kaplan-Meier curve showing prolonged survival of shSELP PD-GB4 tumor-bearing mice compared to control WT and shNC. N = 8 control, 9 shNC, and 11 shSELP. P values were determined using two sided log rank test. C. Representative T1 weighted MRI images of PD-GB4 tumors following Gd-DTPA administration, detected at day 22 post tumor inoculation. Representative images and quantification of immunostaining for proliferating cells (Ki-67), activated microglia (Iba1), and blood vessels (CD31) in the tumors showing reduced proliferation and blood vessel density, and activated microglia in shSELP tumors. Data represent mean ± s.d. N = 5 images per mouse. The graphs show data from a representative mouse per group out of two mice per group. Statistical significance was determined using one-way ANOVA test with multiple comparisons adjustment. Scale bars represent 100 µm. D. Tumor growth of control WT (control), shNC, and shSELP U251 tumors in SCID mice demonstrating delayed tumor growth in shSELP U251 tumors. Tumors were detected by MRI imaging (MR solutions, T2 weighted). Tumor volume was calculated using Radiant software. Data represent mean ± s.e.m. N = 9 control, 8 shNC, and 9 shSELP. One-way ANOVA, Dunn’s method, p < 0.001 E. SELP knockdown prolonged the survival of U251 tumor-bearing mice compared to control WT and shNC. N = 7 control, 6 shNC, and 6 shSELP. P values were determined using two-sided log rank test. F. Representative T2 weighted MRI images of U251 tumors detected at day 19 post tumor inoculation. Representative images and quantification of immunostaining for proliferating cells (Ki-67), activated microglia (Iba1), and blood vessels (CD31) in the tumors showing reduced proliferation and blood vessel density, and activated microglia in shSELP tumors. Data represent mean ± s.d. Each dot represents the average of three images per mouse. N = 3 mice per group. Statistical significance was determined using one-way ANOVA test with multiple comparisons adjustment. Scale bars represent 100 µm.

FIGS. 5A, 5B and 5C. SELP-knockdown inhibits tumor growth and prolongs survival in a murine GB mouse model. A. Tumor growth of control WT (control), shNC, and shSELP GL261 tumors in C57BL/6 mice demonstrating delayed tumor growth in the shSELP group. Tumors were detected by MRI imaging (MR solutions, T1 weighted). Tumor volume was calculated using software. Data represent mean ± s.e.m. N = 29, two independent experiments. One-way ANOVA, Dunn’s method, P < 0.001 B. Kaplan-Meier curve showing a prolonged survival of shSELP GL261 tumor bearing mice compared to control WT and shNC. N = 24 Control, 18 shNC, 15 shSELP, two independent experiments. p values were determined using two-sided log rank test. C. Representative T1 weighted MRI images of GL261 tumors following Gd-DTPA administration, detected at day 19 post tumor inoculation. Representative images and quantification of immunostaining for proliferative cells (Ki-67), apoptotic cells (caspase-3) and blood vessels (CD31). The results demonstrate reduced proliferation, blood vessel density and increased apoptosis in shSELP tumors. Data represent mean ± s.d. Each dot represents the average of three images per mouse. N = 3 mice per group. Statistical significance was determined using one-way ANOVA test with multiple comparisons adjustment. Scale bars represent 100 µm.

FIGS. 6A, 6B, 6C, 6D, 6E, 6F and 6G. Perturbation of the SELP-PSGL-1 interactions causes tumor cells to exhibit reduced tumorigenesis and the microglia/macrophages cells to express higher score of neurodegenerative and antigen presentation signatures. A. Unsupervised clustering of the single-cell RNA profiles of different cell types populations of interest sorted from GL261 tumors. B. Clusters distribution between the shNC (NC) and shSELP groups within the tumor cell population. C. Representative genes from the tumor cells clusters and comparison of gene signature scores showing down-regulation of the invasion, proliferation, and angiogenesis signatures in shSELP tumors compared to shNC. Center of the box plots shows median values, boxes extent from 25% to the 75% percentile, whiskers show minimum and maximum values. Statistical significance was determined using an unpaired, two-sided Student’s t-test. D. Unsupervised clustering of the single-cell RNA profiles of microglia/macrophage cells. E. Bar graphs showing the number of cells present in each cluster from microglia/macrophages isolated from shNC versus shSELP GL261 tumors. F. Projection of a neurodegenerative-associated microglia signature and bar plots showing up-regulation in shSELP tumors compared to shNC (p value = 2.2× 10⁻¹⁶, two-sided Wilcoxon rank sum test and p value = 4.4(×10⁻⁹ CERNO enrichment test, see Methods). G. Projection of an antigen presentation and chemokine/cytokine microglia signature and bar plots showing up-regulation in shSELP tumors compared to shNC (p value = 8.969×10⁻¹⁶, two-sided Wilcoxon rank sum test and p value = 6.4×10⁻⁸ CERNO enrichment test, see Methods). The contour marks the region of highly scored cells by taking into account only cells with a signature score above the 10^{th} percentile.

FIGS. 7A, 7B, 7C, 7D, 7E, 7F, 7G and 7H. SELPi treatment delays the growth of murine and human GB tumors in mice. A. Treatment with SELPi reduced tumor growth of GL261 GB tumors in mice. Representative MRI scanning of day 17 post tumor inoculation. N = 5 mice per group. Data represent mean ± s.e.m. One-way ANOVA, Holm-Sidak’s method, p < 0.009 B. SELPi treatment reduced the proliferation (Ki-67) and microvessel density (CD31) in GL261 GB tumors. Data represent mean ± s.d. Each dot represents the average of three images per mouse. N = 3 mice per group. C. Systemic SELPi treatment reduced tumor growth of iAGR53 tumors in mice Representative images of day 16 scan. P value was calculated by One-way ANOVA test with multiple comparisons adjustment. ‘N’ refers to number of mice per group. Center of the box plots shows median values, boxes extent from 25% to the 75% percentile, whiskers show minimum and maximum values. D. SELPi treatment prolonged the survival of iAGR53 tumor-bearing mice. N = 15 saline, 8 vehicle, and 12 SELPi (mice per group). P values were determined using two-sided log rank test. E. SELPi treatment delayed tumor growth of U251 tumors by SELPi treatment. Data represent mean ± s.e.m. N = 3 saline, 4 vehicle, and 4 SELPi (mice per group). One-way ANOVA, Dunn’s method, p < 0.006 F. H&E staining of U251 tumors. Immunostaining demonstrating reduction in proliferation (Ki-67), activated microglia (Iba1), and blood vessels (CD31) in SELPi treated tumors. Data represent mean ± s.d. Each dot represents the average of three images per mouse. N = 3 mice per group. G. Tumor volume of PD-GB4 tumors in mice demonstrating delayed growth of PD-GB4 tumors by local SELPi treatment. Representative MRI scanning of day 21 post tumor inoculation. Data represent mean ± s.e.m. N = 5 PBS, 3 DMSO, and 5 SELPi. One-way ANOVA, Dunn’s method, p < 0.018. H. H&E staining of PD-GB4 tumors. Immunostaining demonstrating reduction in proliferation (Ki-67), activated microglia (Iba1), and blood vessels (CD31) in SELPi treated tumors. Data represent mean ± s.d. N = 3-4 images per mouse, 2 mice per group. Statistical significance of all the immunostaining was determined using one-way ANOVA test with multiple comparisons adjustment. All Scale bars represent 100 µm.

FIGS. 8A, 8B, 8C, 8D and 8E. Combined treatment of SELPi with GB NV significantly reduced tumor growth, prolonged mice survival and promoted splenocytes activation. A. A scheme showing the treatment regime used for the combined treatment with SELPi and NV. B-C. Tumor volume detected by MRI (T1 weighted, MR Solutions) of GL261 tumors in mice, untreated or treated with free neoantigen peptide, NV, SELPi or SELP + NV. Tumor volume of day 14 (B) and 17 (C) post tumor inoculation are presented. Data represent mean ± s.d. D. Kaplan-Meier curve showing prolonged survival of SELPi + NV treated GL261 tumor bearing mice compared to saline, free peptide, NV or SELPi. N = 8 saline, 8 free peptide, 9 NV, 9 SELPi and 8 SELPi + NV. P values were determined using log rank test. E. mCherry-labeled GL261 spheroids co-cultured with splenocytes isolated from the treated mice. Splenocytes isolated from SELPi+NV treated group showed the strongest ability to reduced spheroids growth. Spheroid growth was monitored for 8 days. Images and analyses were obtained using the IncuCyte imaging system. Data represent mean ± s.d. N=5 spheroids per group.

### DESCRIPTION OF SPECIFIC EMBODIMENTS OF THE INVENTION

The present invention, in some embodiments thereof, relates to a method of treating glioblastoma by selectively decreasing the activity or amount of P-selectin.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present invention is derived from the experimental results presented herein that clearly show that P-selectin plays an important role in GB progression and associated immunosuppressive effects in the brain microenvironment. Using advanced in vitro and ex vivo techniques, as well as pre-clinical human and murine GB mouse models, the present inventors demonstrate that blocking P-selectin has a powerful anti-tumorigenic effect on all these parameters. Using three lenti-induced murine GB cells representing the mesenchymal, proneural and classical GB subtypes, as well as human cell lines and patient-derived GB cells, the present inventors show the relevance of P-selectin-mediated GB-GAMs interactions to the clinical settings.

Thus, according to a first aspect of the present invention there is provided a method of treating glioblastoma in a subject in need thereof comprising administering to the subject a therapeutically effective amount of an agent that specifically decreases an amount and/or activity of P-selectin, thereby treating the glioblastoma.

As used herein, the term “glioblastoma” (GBM), also called glioblastoma multiforme or “grade IV astrocytoma” according to WHO classification refers to a central nervous system primary tumor derived from glial cells. GBM is one of the deadliest human cancers with an incidence of about 3.5/100,000 per year worldwide (Cloughesy, T. F., W. K. Cavenee, and P. S. Mischel, Glioblastoma: from molecular pathology to targeted treatment. Annu Rev Pathol, 2014. 9: p. 1-25). Despite the aggressive standard of care currently used including surgery, chemo- and radiotherapy, the prognosis remains very poor with about 15 months overall survival.

According to a particular embodiment, the glioblastoma is at an early stage (e.g. when the tumor is of a diameter of less than 14 mm).

As used herein, the term “subject” refers to a mammal, such as a rodent, a feline, a canine, and a primate. Preferably, a subject according to the invention is a human. In one embodiment, the subject is a non-operable and non-irradiable subject. In one embodiment, the subject has a tumor comprising two or more lobes.

P-selectin is a member of the selectin family of adhesion glycoproteins which also includes L- and E-selectins. The selectins mediate the recruitment, initial tethering and rolling, and adherence of leukocytes to sites of inflammation. P-selectin is stored in Weibel-Palade bodies of endothelial cells and alpha-granules of platelets and is rapidly mobilized to the plasma membrane upon stimulation by vasoactive substances such as histamine and thrombin.

P-selectin is a transmembrane glycoprotein (SwissProt sequence P16109) composed of an NH₂-terminal lectin domain, followed by an epidermal growth factor (EGF)-like domain and nine consensus repeat domains. It is anchored in the membrane by a single transmembrane domain and contains a small cytoplasmic tail.

Human P-selectin (also referred to as SELP) has a Uniprot number P16109 and a REFSEQ mRNA NM_003005.4.

P-selectin plays its central role in the recruitment of leukocytes to inflammatory and thrombotic sites by binding to its counter-receptor, P-selectin glycoprotein ligand-1 (PSGL-1) (or a PSGL-1-like receptor on sickled red blood cells), which is a mucin-like glycoprotein constitutively expressed on leukocytes including neutrophils, monocytes, platelets, and on some endothelial cells.

Human PSGL-1 has a Uniprot Number Q14242 and REFSEQ mRNA as set forth in NM_001206609.2 or NM_003006.4.

Thus, the present invention contemplates down-regulating the function of P-selectin by using (1) antibodies to P-selectin, (2) antibodies to PSGL-1, (3) small molecules that mimic the binding domain of PSGL-1, and (4) other molecules that disrupt the binding of P-selectin to PSGL-1. Such agents are further described herein below.

In another embodiment, the agent down-regulates expression of P-selectin.

In still another embodiment, the agent down-regulates expression of PSGL-1.

As used herein the phrase “downregulates expression” refers to downregulating the expression of P-selectin or PSGL-1 at the genomic (e.g. homologous recombination and site specific endonucleases) and/or the transcript level using a variety of molecules which interfere with transcription and/or translation (e.g., RNA silencing agents) or on the protein level (e.g., aptamers, small molecules and inhibitory peptides, antagonists, enzymes that cleave the polypeptide, antibodies and the like).

For the same culture conditions, the expression is generally expressed in comparison to the expression in a cell of the same species but not contacted with the agent or contacted with a vehicle control, also referred to as control.

Down regulation of expression may be either transient or permanent.

According to specific embodiments, down regulating expression refers to the absence of mRNA and/or protein, as detected by RT-PCR or Western blot, respectively.

According to other specific embodiments down regulating expression refers to a decrease in the level of mRNA and/or protein, as detected by RT-PCR or Western blot, respectively. The reduction may be by at least a 10%, at least 20%, at least 30%, at least 40%, at least 50%, at least 60%, at least 70%, at least 80%, at least 90%, at least 95% or at least 99% reduction.

Non-limiting examples of agents capable of down regulating P-selectin or PSGL-1 expression are described in details hereinbelow.

### Down-Regulation at the Nucleic Acid Level

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

According to specific embodiments, the downregulating agent is a polynucleotide.

According to specific embodiments, the downregulating agent is a polynucleotide capable of hybridizing to a gene or mRNA encoding P-selectin.

According to specific embodiments, the downregulating agent is a polynucleotide capable of hybridizing to a gene or mRNA encoding PSGL-1.

According to specific embodiments, the downregulating agent directly interacts with P-selectin.

According to specific embodiments, the agent directly binds to P-selectin.

According to specific embodiments, the agent indirectly binds P-selectin (e.g. binds an effector of P-selectin).

According to specific embodiments the downregulating agent is an RNA silencing agent or a genome editing agent.

Thus, downregulation of P-selectin or PSGL-1 can be achieved by RNA silencing.

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g., P-selectin) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA - The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects - see for example [Strat et al., Nucleic Acids Research, 2006, Vol. 34, No. 13 3803-3810; Bhargava A et al. Brain Res. Protoc. 2004;13:115-125; Diallo M., et al., Oligonucleotides. 2003;13:381-392; Paddison P.J., et al., Proc. Natl Acad. Sci. USA. 2002;99:1443-1448; Tran N., et al., FEBS Lett. 2004;573:127-134].

According to some embodiments of the invention, dsRNA is provided in cells where the interferon pathway is not activated, see for example Billy et al., PNAS 2001, Vol 98, pages 14428-14433. and Diallo et al., Oligonucleotides, Oct. 1, 2003, 13(5): 381-392. doi:10.10891154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small interfering RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., a siRNA) may be connected to form a hairpin or stem-loop structure (e.g., a shRNA). Thus, as mentioned, the RNA silencing agent of some embodiments of the invention may also be a short hairpin RNA (shRNA).

miRNA and miRNA mimics - According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Antisense - Antisense is a single stranded RNA designed to prevent or inhibit expression of a gene by specifically hybridizing to its mRNA. Downregulation of a P-selectin can be effected using an antisense polynucleotide capable of specifically hybridizing with an mRNA transcript encoding P-selectin. Downregulation of PSGL-1 can be effected using an antisense polynucleotide capable of specifically hybridizing with an mRNA transcript encoding PSGL-1.

Design of antisense molecules, which can be used to efficiently downregulate a P-selectin or PSGL-1must be effected while considering two aspects important to the antisense approach. The first aspect is delivery of the oligonucleotide into the cytoplasm of the appropriate cells, while the second aspect is design of an oligonucleotide, which specifically binds the designated mRNA within cells in a way which inhibits translation thereof.

Downregulation can be achieved by inactivating the gene (i.e. the P-selectin or PSGL-1 gene) via introducing targeted mutations involving loss-of function alterations (e.g. point mutations, deletions and insertions) in the gene structure.

As used herein, the phrase “loss-of-function alterations” refers to any mutation in the DNA sequence of a gene which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments, loss-of-function alteration of a gene comprises both alleles of the gene. In such instances, the P-selectin or PSGL-1 may be in a homozygous form or in a heterozygous form.

Methods of introducing nucleic acid alterations to a gene of interest are well known in the art [see for example Menke D. Genesis (2013) 51: - 618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8771945, 8586526, 6774279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases - this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases - Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. 8,021,867). Meganucleases can be designed using the methods described in e.g., Certo, MT et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8, 148,098; or 8,163,514, the contents of each are incorporated herein by reference in their entirety. Alternatively, meganucleases with site specific cutting characteristics can be obtained using commercially available technologies e.g., Precision Biosciences’ Directed Nuclease Editor™ genome editing technology.

ZFNs and TALENs - Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the non-homologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2- His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May;30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, CA).

CRISPR-Cas system - Many bacteria and archaea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab’s Target Finder, the Michael Boutros lab’s Target Finder (E-CRISP), the RGEN Tools: Cas-OFFinder, the CasFinder: Flexible algorithm for identifying specific Cas9 targets in genomes and the CRISPR Optimal Target Finder.

Non-limiting examples of gRNA sequences that can be used with some embodiments of the present invention are described in the literature (Sanjana N.E., Shalem O., Zhang F. Nat Methods. 2014 Aug;11(8):783-4) and in the genscript website see www(dot)genscriptdotcom/gRNA-detail/6403/SELP-CRISPR-guide-RNA.

According to specific embodiments, the gRNA sequence does not have a significant off target effect. Methods of determining off target effect are well known in the art, such as BGI Human Whole Genome Sequencing (described in Nature;491:65-56.2012), next generation sequencing (NGS) using e.g. commercially available kits such as Alt-R-Genom Editing (IDT detection kit) or Sure select target enrich <1% variant allele frequency (Agilent).

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene. Alternatively, the target cell can be transfected with both gRNA and Cas9 without plasmid using e.g. a transfection reagent such as CRISPRMAX [see e.g. Yu et al. (2016) JDlBiotechnol Lett. 38(6):919-29]. In some cells electroporation can improve the transfection of the gRNA and the Cas9 [see e.g. Liang et al. (2015) Journal of Biotechnology 208, 2015, Pages 44-53; and Liang et al. (2017) Journal of Biotechnology, Volume 241, 2017, pp. 136-146].

“Hit and run” or “in-out” - involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy - involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases - The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases - As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. Dec 1, (2003) 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas Tol2 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform - this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

It will be appreciated that the agent can be a mutagen that causes random mutations and the cells exhibiting downregulation of the expression level and/or activity of the target may be selected.

The mutagens may be, but are not limited to, genetic, chemical or radiation agents. For example, the mutagen may be ionizing radiation, such as, but not limited to, ultraviolet light, gamma rays or alpha particles. Other mutagens may include, but not be limited to, base analogs, which can cause copying errors; deaminating agents, such as nitrous acid; intercalating agents, such as ethidium bromide; alkylating agents, such as bromouracil; transposons; natural and synthetic alkaloids; bromine and derivatives thereof; sodium azide; psoralen (for example, combined with ultraviolet radiation). The mutagen may be a chemical mutagen such as, but not limited to, ICR191, 1,2,7,8-diepoxy-octane (DEO), 5-azaC, N-methyl-N-nitrosoguanidine (MNNG) or ethyl methane sulfonate (EMS).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

### Down-Regulation at the Polypeptide Level

According to specific embodiments the agent capable of downregulating P-selectin is an antibody or antibody fragment capable of specifically binding and inhibiting P-selectin.

Preferably, the antibody specifically binds at least one epitope of P-selectin.

In another embodiment, the agent is an antibody or antibody fragment capable of specifically binding and inhibiting PSGL-1.

Preferably, the antibody specifically binds at least one epitope of PSGL-1.

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three-dimensional structural characteristics, as well as specific charge characteristics.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

The term “antibody” as used in this invention includes intact molecules as well as functional fragments thereof (that are capable of binding to an epitope of an antigen).

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three-dimensional structural characteristics, as well as specific charge characteristics.

According to a specific embodiment, the antibody fragments include, but are not limited to, single chain, Fab, Fab′ and F(ab′)₂ fragments, Fd, Fcab, Fv, dsFv, scFvs, diabodies, minibodies, nanobodies, Fab expression library or single domain molecules such as VH and VL that are capable of binding to an epitope of the antigen in an HLA restricted manner.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), an Fab, an Fab′, and an F(ab′)2, or antibody fragments comprising the Fc region of an antibody.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular’s AbM antibody modeling software (now Accelrys®, see, Martin et al., 1989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 1996) and the “conformational definition” (see, e.g., Makabe et al., Journal of Biological Chemistry, 283:1156-1166, 2008).

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:
(i) Fv, defined as a genetically engineered fragment consisting of the variable region of the light chain (VL) and the variable region of the heavy chain (VH) expressed as two chains;
(ii) single chain Fv (“scFv”), a genetically engineered single chain molecule including the variable region of the light chain and the variable region of the heavy chain, linked by a suitable polypeptide linker as a genetically fused single chain molecule.
(iii) disulfide-stabilized Fv (“dsFv”), a genetically engineered antibody including the variable region of the light chain and the variable region of the heavy chain, linked by a genetically engineered disulfide bond.
(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;
(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);
(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds);
(vii) Single domain antibodies or nanobodies are composed of a single VH or VL domains which exhibit sufficient affinity to the antigen; and
(viii) Fcab, a fragment of an antibody molecule containing the Fc portion of an antibody developed as an antigen-binding domain by introducing antigen-binding ability into the Fc region of the antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Exemplary methods for generating antibodies employ induction of in-vivo production of antibody molecules, screening of immunoglobulin libraries (Orlandi D.R. et al., 1989. Proc. Natl. Acad. Sci. U.S.A. 86:3833-3837; Winter G. et al., 1991. Nature 349:293-299) or generation of monoclonal antibody molecules by continuous cell lines in culture. These include, but are not limited to, the hybridoma technique, the human B-cell hybridoma technique, and the Epstein-Barr virus (EBV)-hybridoma technique (Kohler G. et al., 1975. Nature 256:495-497; Kozbor D. et al., 1985. J. Immunol. Methods 81:31-42; Cote RJ. et al., 1983. Proc. Natl. Acad. Sci. U. S. A. 80:2026-2030; Cole SP. et al., 1984. Mol. Cell. Biol. 62:109-120).

In cases where target antigens are too small to elicit an adequate immunogenic response when generating antibodies in-vivo, such antigens (haptens) can be coupled to antigenically neutral carriers such as keyhole limpet hemocyanin (KLH) or serum albumin [e.g., bovine serum albumin (BSA)] carriers (see, for example, US. Pat. Nos. 5,189,178 and 5,239,078]. Coupling a hapten to a carrier can be effected using methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill. The resulting immunogenic complex can then be injected into suitable mammalian subjects such as mice, rabbits, and the like. Suitable protocols involve repeated injection of the immunogen in the presence of adjuvants according to a schedule which boosts production of antibodies in the serum. The titers of the immune serum can readily be measured using immunoassay procedures which are well known in the art.

The antisera obtained can be used directly or monoclonal antibodies may be obtained as described hereinabove.

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

As described hereinabove, Fv fragments comprise an association of VH and VL chains. This association may be noncovalent, as described in Inbar et al. [Proc. Nat′l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

As mentioned, the antibody fragment may comprise a Fc region of an antibody termed “Fcab”. Such antibody fragments typically comprise the CH2-CH3 domains of an antibody. Fcabs are engineering to comprise at least one modification in a structural loop region of the antibody, i.e. in a CH3 region of the heavy chain. Such antibody fragments can be generated, for example, as follows: providing a nucleic acid encoding an antibody comprising at least one structural loop region (e.g. Fc region), modifying at least one nucleotide residue of the at least one structural loop regions, transferring the modified nucleic acid in an expression system, expressing the modified antibody, contacting the expressed modified antibody with an epitope, and determining whether the modified antibody binds to the epitope. See, for example, U.S. Pat. Nos. 9,045,528 and 9,133,274 incorporated herein by reference in their entirety.

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

The antibodies described herein may be conjugated to a therapeutic moiety. The therapeutic moiety can be, for example, a cytotoxic moiety, a toxic moiety, a cytokine moiety and a second antibody moiety comprising a different specificity to the antibodies of the invention.

Non-limiting examples of therapeutic moieties which can be conjugated to the antibody of the invention are provided in Table 1, hereinbelow.

TABLE 1

| | | |
|---|---|---|
| Therapeutic moiety | Amino acid sequence (GenBank Accession No.) | Nucleic acid sequence (GenBank Accession No.) |
| Pseudomonas exotoxin | ABU63124 | EU090068 |
| Diphtheria toxin | AAV70486 | AY820132.1 |
| interleukin 2 | CAA00227 | A02159 |
| CD3 | P07766 | X03884 |
| CD16 | NP_000560.5 | NM_000569.6 |
| interleukin 4 | NP_000580.1 | NM_000589.2 |
| HLA-A2 | P01892 | K02883 |
| interleukin 10 | P22301 | M57627 |
| Ricin toxin | EEF27734 | EQ975183 |

Other therapeutic moieties that may be attached to the antibody of the invention include anticancer agents such as chemotherapeutic agents including but not limited to tubulin inhibitors, such as exatecan, belotecan, Emtansine, etc.

The therapeutic moiety may be attached or conjugated to the antibody of the invention in various ways, depending on the context, application and purpose.

When the functional moiety is a polypeptide, the immunoconjugate may be produced by recombinant means. For example, the nucleic acid sequence encoding a toxin (e.g., PE38KDEL) or a fluorescent protein [e.g., green fluorescent protein (GFP), red fluorescent protein (RFP) or yellow fluorescent protein (YFP)] may be ligated in-frame with the nucleic acid sequence encoding the antibody of the invention and be expressed in a host cell to produce a recombinant conjugated antibody. Alternatively, the functional moiety may be chemically synthesized by, for example, the stepwise addition of one or more amino acid residues in defined order such as solid phase peptide synthetic techniques.

A functional moiety may also be attached to the antibody of the invention using standard chemical synthesis techniques widely practiced in the art [see e.g., hypertexttransferprotocol://worldwideweb (dot) chemistry (dot) org/portal/Chemistry)], such as using any suitable chemical linkage, direct or indirect, as via a peptide bond (when the functional moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like. Description of fluorescent labeling of antibodies is provided in details in U.S. Pat. Nos. 3,940,475, 4,289,747, and 4,376,110.

Exemplary methods for conjugating peptide moieties (therapeutic or detectable moieties) to the antibody of the invention are described herein below:

SPDP conjugation - A non-limiting example of a method of SPDP conjugation is described in Cumber et al. (1985, Methods of Enzymology 112: 207-224). Briefly, a peptide, such as a detectable or therapeutic moiety (e.g., 1.7 mg/ml) is mixed with a 10-fold excess of SPDP (50 mM in ethanol); the antibody is mixed with a 25-fold excess of SPDP in 20 mM sodium phosphate, 0.10 M NaCl pH 7.2 and each of the reactions is incubated for about 3 hours at room temperature. The reactions are then dialyzed against PBS. The peptide is reduced, e.g., with 50 mM DTT for 1 hour at room temperature. The reduced peptide is desalted by equilibration on G-25 column (up to 5 % sample/column volume) with 50 mM KH₂PO₄ pH 6.5. The reduced peptide is combined with the SPDP-antibody in a molar ratio of 1:10 antibody:peptide and incubated at 4° C. overnight to form a peptide-antibody conjugate.

Glutaraldehyde conjugation - A non-limiting example of a method of glutaraldehyde conjugation is described in G.T. Hermanson (1996, “Antibody Modification and Conjugation, in Bioconjugate Techniques, Academic Press, San Diego). Briefly, the antibody and the peptide (1.1 mg/ml) are mixed at a 10-fold excess with 0.05 % glutaraldehyde in 0.1 M phosphate, 0.15 M NaCl pH 6.8, and allowed to react for 2 hours at room temperature. 0.01 M lysine can be added to block excess sites. After-the reaction, the excess glutaraldehyde is removed using a G-25 column equilibrated with PBS (10 % v/v sample/column volumes).

Carbodiimide conjugation - Conjugation of a peptide with an antibody can be accomplished using a dehydrating agent such as a carbodiimide, e.g., in the presence of 4-dimethyl aminopyridine. Carbodiimide conjugation can be used to form a covalent bond between a carboxyl group of peptide and an hydroxyl group of an antibody (resulting in the formation of an ester bond), or an amino group of an antibody (resulting in the formation of an amide bond) or a sulfhydryl group of an antibody (resulting in the formation of a thioester bond). Likewise, carbodiimide coupling can be used to form analogous covalent bonds between a carbon group of an antibody and an hydroxyl, amino or sulfhydryl group of the peptide [see, J. March, Advanced Organic Chemistry: Reaction’s, Mechanism, and Structure, pp. 349-50 & 372-74 (3d ed.), 1985]. For example, the peptide can be conjugated to an antibody via a covalent bond using a carbodiimide, such as dicyclohexylcarbodiimide [B. Neises et al. (1978), Angew Chem., Int. Ed. Engl. 17:522; A. Hassner et al. (1978, Tetrahedron Lett. 4475); E.P. Boden et al. (1986, J. Org. Chem. 50:2394) and L.J. Mathias (1979, Synthesis 561)].

According to another embodiment, the antibodies described herein are not conjugated to a therapeutic or a diagnostic moiety.

Another agent which can be used along with some embodiments of the invention to downregulate P-selectin is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

Another agent capable of downregulating P-selectin would be any molecule which binds to and/or cleaves P-selectin. Such molecules can be a small molecule, P-selectin antagonists, or P-selectin inhibitory peptide.

Another contemplated agent which can be used to downregulate P-selectin includes a proteolysis-targeting chimaera (PROTAC). Such agents are heterobifunctional, comprising a ligand which binds to a ubiquitin ligase (such as E3 ubiquitin ligase) and a ligand to P-selectin and optionally a linker connecting the two ligands. Binding of the PROTAC to the target protein leads to the ubiquitination of an exposed lysine on the target protein, followed by ubiquitin proteasome system (UPS)-mediated protein degradation.

In an embodiment, the P-selectin inhibitor is a monoclonal antibody directed towards P-selectin, such as crizanlizumab or inclacumab. These antibodies against P-selectin have been developed to treat sickle cell anemia and myocardial damage following a heart attack, respectively. Both antibodies were well tolerated in patients when administered systemically.

According to a particular embodiment, the P-selectin inhibitor is a monoclonal antibody directed towards PSLGL-1. An example of such an antibody is VTX-0811, which is being developed by Verseau therapeutics (www(dot)verseautxdotcom/pipeline).

In another embodiment, the P-selectin inhibitor is a small molecule such as rivipansel or tinzaparin, which have been developed to treat sickle cell anemia and as an anticoagulant, respectively. Rivipansel is not specific to P-selectin, but inhibits several members of the selectins family. Tinzaparin is a heparin analogue.

In another embodiment, the P-selectin inhibitor is KF 38789 manufactured by Tocris (3-[7-(2,4-Dimethoxyphenyl)-2,3,6,7-tetrahydro-1,4-thiazepin-5-yl]-4-hydroxy-6-methyl-2H-pyran-2-one).

Additional exemplary P-selectin inhibitors are summarized in Table 2, herein below.

TABLE 2

| | | | | |
|---|---|---|---|---|
| Product name | Product description | Purpose | Phase | References |
| Crizanlizumab (SelGl, Adakveo) | Humanized monoclonal anti-P-selectin antibody. | Treatment of retinal vasculopathy with cerebral leukoencephalopathy (RVCL) | II | NCT04611880 [1] |
| | | Study to assess safety and impact of SelG1 with or without Hydroxyurea therapy in Sickle Cell Disease patients with pain Crises. | II | NCT01895361 |
| | | Crizanlizumab for treating COVID-19 vasculopathy (CRITICAL). | II | NCT04435184 |
| PSI-697 | Oral P-selectin inhibitor. 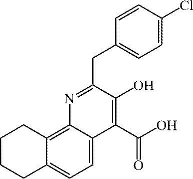 [SMILES: O=C(O)c1c(O)c(Cc2ccc(Cl)cc2)nc2c3c(ccc12)CCCC3] | Study evaluating the effects of PSI-697 on platelets in subjects who smoke. | I | NCT03860506 [2] |
| Cylexin (CY-1503) | Synthetic analogue of sialyl Lewis X oligosaccharide. 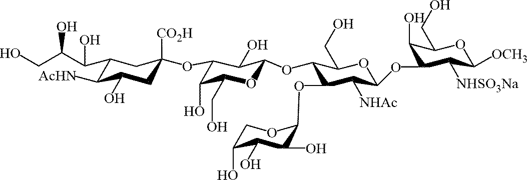 | Cylexin for reduction of reperfusion injury in infant heart surgery. | II, III | NCT00226369 [3] |
| Bimosiamose (TBC1269) | Low-molecular weight sLex-blocking selectin inhibitor that has been shown to inhibit P-selectin-, E-selectin-, and L-selectin-dependent adhesion in humans in vitro and to exert anti-inflammatory effects in different animal models. | Study to evaluate safety and efficacy of inhaled Bimosiamose for the treatment of patients with moderate to severe chronic | II | NCT01108913 [4] |
| | 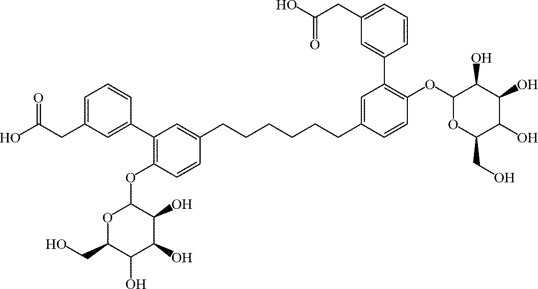 [SMILES: O=C(O)Cc1cccc(-c2cc(CCCCCCc3ccc(OC4O[C@H](CO)C(O)[C@H](O)[C@@H]4O)c(-c4cccc(CC(=O)O)c4)c3)ccc2OC2O[C@H](CO)C(O)[C@H](O)[C@@H]2O)c1] | obstructive pulmonary disease (COPD). | | |
| | | Study to evaluate the effect of bimosiamose on ozone induced sputum Neutrophilia. | II | NCT00962481 |
| | | Safety and efficacy study of Bimosiamose Cream to treat psoriasis. | II | NCT00823693 |
| Rivipansel (GMI-1070) | Novel small molecule glycomimetic pan-selectin antagonist. 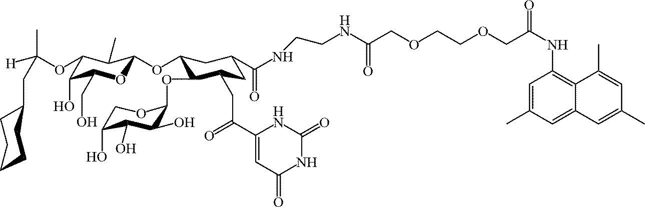 | Study of Intravenous GMI-1070 in adults with Sickle Cell Disease. | II, III | NCT00911495 [5] |
| | | Study of GMI-1070 for the treatment of Sickle Cell Pain Crisis. | II | NCT01119833 |

1. Ataga, K.I., et al., Crizanlizumab for the Prevention of Pain Crises in Sickle Cell Disease. N Engl J Med, 2017. 376(5): p. 429-439.
2. Bedard, P.W., et al., Characterization of the novel P-selectin inhibitor PSI-697 [2-(4-chlorobenzyl)-3-hydroxy-7,8,9,10-tetrahydrobenzo[h] quinoline-4-carboxylic acid] in vitro and in rodent models of vascular inflammation and thrombosis. J Pharmacol Exp Ther, 2008. 324(2): p. 497-506.
3. Park, I.Y., et al., Cylexin: a P-selectin inhibitor prolongs heart allograft survival in hypersensitized rat recipients. Transplant Proc, 1998. 30(7): p. 2927-8.
4. Mayr, F.B., et al., Effects of the pan-selectin antagonist bimosiamose (TBC1269) in experimental human endotoxemia. Shock, 2008. 29(4): p. 475-82.
5. Chang, J., et al., GMI-1070, a novel pan-selectin antagonist, reverses acute vascular occlusions in sickle cell mice. Blood, 2010. 116(10): p. 1779-86.

The agent which is used to down-regulate the amount and/or activity of P-selectin may be formulated for crossing the blood brain barrier.

Exemplary methods for formulating the above described agents to enhance its penetration across the blood brain barrier are described in Yeini et al., Advanced Therapeutics, DOI: 10.1002/adtp.202000124.

Thus, for example, the agents can be formulated in nanoparticles such as liposome-based nanoparticles, amphiphilic micelles, dendrimers, inorganic nanoparticles and polymeric nanoparticles.

Specifically for the delivery of oligonucleotides, the use of cationic nanoemulsions modified biodegradable poly(β-Amino Ester) (PBAE), cell derived extracellular vesicles, spherical nucleic acid nanoparticles, may be considered to improve delivery to the brain.

Since the BBB restricts the passage of most therapeutic agents from the blood to the brain, receptor-mediated transcytosis can offer a non-invasive trafficking system to deliver targeted carriers into the brain parenchyma. In addition, this approach allows selective targeting of tumor cells within the brain tissue, thus reducing toxicity in other tissues and non-tumor cells in the brain. Examples of receptor-mediated approaches include manipulation of the apolipoprotein receptor, targeting of the epidermal growth factor receptor, transferrin receptor targeting, insulin receptor targeting and adhesion molecule targeting are all contemplated.

It will be appreciated that the P-selectin inhibitory agents may be directly attached to moieties that target the agent to the blood brain barrier or indirectly (e.g. P-selectin inhibitory agents may be comprised in a carrier which may be attached to the targeting moieties).

The P-selectin inhibitors of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the P-selectin inhibitor accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington’s Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into the brain of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank’s solution, Ringer’s solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multi-dose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (P-selectin inhibitor) effective to prevent, alleviate or ameliorate symptoms of a disorder (e.g., P-selectin inhibitor) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient’s condition. (See e.g., Fingl, et al., 1975, in “The Pharmacological Basis of Therapeutics”, Ch. 1 p.1).

Dosage amount and interval may be adjusted individually to provide brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

According to a particular embodiment, the active agent is administered following resection of said glioblastoma tumor.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

### Method of Treating

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

### Treatment Regimen

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relief symptoms of the pathology yet results in incomplete cure of the pathology. It will be appreciated that in certain cases the more aggressive treatment regimen may be associated with some discomfort to the subject or adverse side effects (e.g., a damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

As mentioned, the P-selectin inhibitor may be administered/co-formulated with an immunomodulatory agent. In one embodiment, the immunomodulatory agent is a nanoparticle which comprises a neoantigen peptide of glioblastoma (e.g. GL261). Methods of formulating such nanoparticles are disclosed in WO2020/136657, the contents of which are incorporated herein by reference.

Examples of immunomodulatory agents include immunomodulatory cytokines, including but not limited to, IL-2, IL-15, IL-7, IL-21, GM-CSF as well as any other cytokines that are capable of further enhancing immune responses; immunomodulatory antibodies, including but not limited to, anti-CTLA4, anti-CD40, anti-41BB, anti-OX40, anti-PD1 and anti-PDL1; and immunomodulatory drugs including, but not limited to lenalidomide (Revlimid).

As used herein the term “about” refers to ± 10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization - A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### MATERIALS AND METHODS

DMEM, fetal bovine serum (FBS), L-glutamine, penicillin, streptomycin, mycoplasma detection kit, EZ-RNA II total RNA isolation kit and fibronectin (1 mg/ml, dilution: 1:100) were purchased from Biological Industries Ltd. (Kibbutz Beit HaEmek, Israel). Percoll medium (Cat. No. p4937) and all other chemical reagents, including salts and solvents, were purchased from Sigma-Aldrich (Rehovot, Israel). Milli-Q water was prepared using a Millipore water purification system. Amicon Ultra Centrifugal Filters; molecular weight cut-off (MWCO) 5 or 3 kDa and Poly-L-Lysine (PLL) (Cat. No. A-005-C; 0.1 mg/ml) were purchased from Merck Millipore (Burlington, Massachusetts, USA). The qScript™ cDNA Synthesis Kit was purchased from Quantabio (Beverly, MA, USA). Fast SYBR™ green Master Mix was purchased from Applied Biosystems (California, USA). Collagenase IV, Dispase II (neutral protease) and DNase I were purchased from Worthington Biochemical Corporation (NJ, USA). RBC lysis solution (Cat. No. 420301) was purchased from BioLegend (San Diego, California, USA). MACS MS magnetic columns for cell separation (Cat. No. 130-042-201), CD11b MicroBeads for cell isolation (Cat. No. 130-093-634) and CD45 (TIL) MicroBeads for cell isolation (Cat. No. 130-110-618) were purchased from Miltenyi Biotec (Bergisch Gladbach, Germany). SELP inhibitor (SELPi) KF38789 (Cat. No. 2748) was purchased from Tocris BioScience (Bristol, United Kingdom). Recombinant human SELP (Cat. No. ADP3; Lot. No. ARL6019071), Recombinant murine SELP (rSELP) (Cat. No. 10094-PS; Lot. No. DKLJ0118111), Human SELP ELISA kit (Cat. No. DPSE00), Total NO/Nitrite/Nitrate Immunoassay (Cat. No. KGE001), Mouse XL Cytokine Array Kit (Cat. No. ARY028), Human Cytokine Array kit (Cat. No. ARY005B), anti-human PSGL-1 neutralizing antibody (Cat. No. MAB3345; Lot. No. CLYK0120111; Clone 688102) and anti-human SELP neutralizing antibody (Cat. No. BBA1; Lot. No. APB081704; Clone BBIG-E) were purchased from R&D Systems (Minneapolis, Minnesota, USA). Human L-507 cytokine array kit (Cat. No. AAH-BLM-1A-4; Lot No. 102920 009) was purchased from RayBiotech (Norcross, Georgia, United States). Anti-human/mouse CD44 neutralizing antibody (Cat. No. NBP2-2530; Lot No. VC289186) was purchased from Novus (Colorado, USA). Anti-murine PSGL-1 neutralizing antibody (Cat. No. BE0188; Lot No. 676818M2) was purchased from Bio X Cell (Massachusetts, USA). MEBCYTO Apoptosis Kit, was purchased from MBL International (UK), Recombinant murine GM-CSF (Cat. No. 315-03-50ug; Lot. No. 091855) was purchased from PeproTech (Rehovot, Israel). Latex beads for phagocytosis assays (Cat. No. L4655) were purchased from Sigma-Aldrich (Rehovot, Israel). ProLong® Gold mounting with DAPI (Cat. No. p36935) and Hoechst 33342 (Cat. No. H3570) were purchased from Invitrogen (Carlsbad, California, USA). Mayer’s Hematoxylin solution (Cat. No. 05-06002) and Eosin Y solution (Cat. No. 05-10002) were purchased from Bio-Optica (Milano, Italy).

Plasmids: mCherry was subcloned by our group into the pQCXIP vector (Clontech, USA) as previously described [37]. iRFP was used as previously described [38]. Human SELP shRNA (Cat. No. sc-29421-SH) and human negative control (NC) shRNA (Cat. No. sc-108060) containing plasmids were purchased from Santa Cruz Biotechnology, Inc. (Dallas, Texas, USA). Murine SELP shRNA and murine NC shRNA plasmids (Simple hairpin shRNAs in the pLKO.1 lentiviral vector designed by The RNAi Consortium) were purchased from GE Healthcare Dharmacon, Inc. (Lafayette, Colorado, USA). Anti-human (SWA11) and anti-murine (M1.69) CD24 neutralizing antibodies were kindly provided by Nadir Arber and Shiran (Tel Aviv Sourasky Medical Center).

Primary immunostaining antibodies: Rabbit anti-mouse Iba1 (Cat. No. NBP2-19019; Lot. No. 41556; Dilution: 1:200), rat anti-human/mouse PSGL-1 (Cat. No. NB100; Lot. No. C; Dilution 1:50), rabbit anti-human/mouse Ki-67 (Cat. No. NB500-170; Lot. No. G15; Dilution 1:50), and rabbit anti-mouse FOXP3 (Cat. No. NB600; Lot. No. D-1; Dilution 1:30) were purchased from Novus (Colorado, USA). Mouse anti-human SELP (Cat. No. BBA1; Lot. No. APB081704; Clone BBIG-E; Dilution 1:30) was purchased from R&D Systems (Minneapolis, Minnesota, USA). Mouse anti-mouse SELP (Cat. No. 148302; Lot No. B186735; Clone RMP-1; Dilution 1:50) was purchased from BioLegend (San Diego, California, USA). Rat anti-mouse CD31 (Cat. No. 550272; Lot. No. 6273859; Dilution 1:25) was purchased from BD Biosciences (Franklin Lakes, NJ, USA). Rabbit anti-human/mouse Caspase-3 (Cat. No. CST-9664L; Lot. No. 21; Dilution 1:30) was purchased from Cell Signaling Technology (Danvers, Massachusetts, USA). Rat anti-mouse CD4 (Cat. No. 14-9766-82; Lot. No. 4307664; Clone 4SMAS; Dilution 1:100) and Rat anti-CD8 (Cat. No. 14-0808-82; Lot. No. 2003225; Clone: 4SMIS; Dilution 1:50) were purchased from eBioscience (San Diego, California, USA).

Secondary immunostaining antibodies: Goat anti-mouse Alexa Fluor® 647 (Cat. No. ab15115; Lot. No. GR309891-3; Dilution 1:300), goat anti-rabbit Alexa Fluor® 488 (Cat. No. ab150077; Lot No. GR315933-2; Dilution 1:300), and goat anti-rabbit Alexa Fluor® 647 (Cat. No. Ab150079; Lot. No. Gr3176223-2; Dilution 1:300) were purchased from Abcam (Cambridge, United Kingdom). Goat anti-rat Alexa Fluor® 488 (Cat 112-545-068; Lot. No. 143654; Dilution 1:300) and goat anti-rat Alexa Fluor® 647 (Cat. No. 112-605-003; Lot No. 137652; Dilution 1:300) were purchased from Jackson ImmunoResearch Laboratories, Inc. (West Grove, Pennsylvania, USA).

Flow cytometry antibodies: Mouse anti-human SELP (Cat. No. BBA1; Lot. No. APB081704; Clone BBIG-E; Dilution 1:20), Mouse IgG1 isotype control (Cat. No. mab002; Dilution 1:20) were purchased from R&D Systems (Minneapolis, Minnesota, USA). Rat anti-human/mouse PSGL-1 (Cat. No. NB100; Lot. No. C; Dilution 1:24) was purchased from Novus (Colorado, USA). Rabbit anti-human/mouse CD163 (Cat. No. AB182422; Lot. No. GR3232711-5; Dilution 1:20), anti-mouse CD11b (Cat. No. ab8878; Lot. No. GR131048-4; Dilution 1:25), and goat anti-rabbit Alexa Fluor® 488 (Cat. No. ab150077; Lot No. GR315933-2; Dilution 1:50) were purchased from Abcam (Cambridge, United Kingdom). Anti-mouse CD3-FITC (Cat. No. 130-119-798; Lot. No. 5190919162; Clone REA641; Dilution 1:10), anti-mouse CD8-APC (Cat. No. 130-111-712; Lot No. 5190919051; Clone: REA793; Dilution 1:10), anti-mouse CD4 VioBlue® (Cat. No. 130-118-696; Lot No. 5190919087; Clone REA605; Dilution 1:10), REA Control-APC (Cat. No. 130-113-446; Lot. No. 5190711317; Clone REA293; Dilution 1:10), REA Control-VioBlue® (Cat. No. 130-113-545; Lot. No. 5190711335; Clone REA293; Dilution 1:10), anti-mouse CD38-APC-Vio770 (Cat. No. 130-125-227; Lot No. 5200405654; Clone REA616; Dilution 1:50), anti-mouse F4/80-FITC (Cat. No. 130-117-509; Lot. Np. 52003066886; Clone REA126; Dilution 1:50), anti-CD11b-PE-Vio770 (Cat No. 130-113-808; Lot No. 5190919070; Clone REA592; Dilution 1:50) and REA Control-FITC (Cat. No. 130-113-449; Lot No. 5190711318; Clone REA293; Dilution 1:10) were purchased from Miltenyi Biotec (Bergisch Gladbach, Germany). Anti-mouse FOXP3 Alexa Fluor® 647 (Cat. No. 126408; Lot. No. B264076; Clone MF-14; Dilution 1:25), Alexa Fluor® 647 IgG2b, k Isotype ctrl (Cat. No. 400626; Lot. No. B243822; Clone RTK4530; Dilution 1:25), anti-mouse P2Y12-PE (Cat. No. 848003; Lot No. B264216; Clone S16007D, Dilution 1:50), anti-human P2Y12-Briliant Violet 421 (Cat No. 392105; Lot No. B286137; Clone S16001E; Dilution 1:50), anti-human TMEM119 (Cat No. 853301; Lot No. B272769; Clone A16075D; Dilution 1:50), anti-mouse CD206-PE (Cat. No. 141706; Lot No. B280038; Clone C068C2; Dilution 1:50) and LEAF Purified Rat IgG2b, k Isotype Ctrl (Cat. No. 400621; Lot. No. B209798; Clone RTK4530) were purchased from BioLegend (San Diego, California, USA). Mouse-IgG k BP-CFL 488 (Cat. No. sc-516176; Lot. No. HO118; Dilution 1:20) was purchased from Santa Cruz Biotechnology, Inc. (Dallas, Texas, USA). Goat anti-rat Alexa Fluor® 488 (Cat 112-545-068; Lot. No. 143654; Dilution 1:50) was purchased from Jackson ImmunoResearch Laboratories, Inc. (West Grove, Pennsylvania, USA). anti-human CD206 (Cat No. 60143-1-ig; Dilution 1:100) and anti-human TMEM119 (Cat. No. 27585-1-ap; Dilution 1:100) were purchased from ProteinTech (Rosemont, California, USA).

Western Blot antibodies: Anti-human total p65 (Cat. No. D14E12; Lot. No. 13; Dilution 1:1000), anti-human phospho p65 (Cat. No. S536; Lot. No. 16; Dilution 1:1000), anti-human vinculine (Cat. No. E1E9V; Lot. No. 6; Dilution 1:1000) and anti-rabbit HRP (Cat. No. 7074P2; Lot. No. 28; Dilution 1:2000) were purchased from Cells Signaling (Massachusetts, USA)

Cell culture. U251 human GB cell line was obtained from the European Collection of Authenticated Cell Cultures (ECACC) (Porton Down, Salisbury, UK). GL261 murine GB cell line was obtained from the National Cancer Institute (Frederick, MD, USA). The mesenchymal iAGR53, the proneural PNp53 and the classical EGFRviii-shP16 murine GB cell lines were prepared as previously described [39, 40]. Cells were cultured in Dulbecco’s modified Eagle’s medium (DMEM) supplemented with 10% FBS, 100 U/ml Penicillin, 100 µg/ml Streptomycin, 12.5 U/ml Nystatin, 2 mM L-glutamine (Biological Industries, Israel). Primary human microglia were obtained from Celprogen (Torrance, California, USA) and cultured in microglia medium (Celprogen) supplemented with 10% FBS. Cells were routinely tested for mycoplasma contamination with a mycoplasma detection kit (Biological Industries, Israel). All cells were grown at 37° C. in 5% CO₂.

Human primary GB cells. Patient-derived GB cells (PD-GB) were isolated from clinical samples obtained from surgical procedures. Tumor tissues were kept in cold PBS and processed within 40 min. In order to isolate tumor cells, tumor samples were dissected to 0.5 mm pieces, and were then plated in 6 cm plates, and cultured in 1 ml DMEM supplemented with 10% FBS, 100 U/ml Penicillin, 100 mg/ml Streptomycin, and 2 mM L-glutamine. Viable cancer cells remained attached to culture plates during medium changes and kept growing in culture, while stromal cells and cell debris were washed away. Cells were routinely tested for mycoplasma contamination with a mycoplasma detection kit (Biological Industries, Israel). All cells were grown at 37° C. in 5% CO₂.

Primary murine brain cell isolation. Brains resected from healthy, 5-8 week old C57/BL6 mice, were chopped and incubated with 1 mg/ml Collagenase IV, 2 mg/ml Dispase II (neutral protease), and 0.02 U/ml DNase I for 50 min at 37° C. Red blood cells (RBC) were lysed with RBC lysis solution followed by a Percoll gradient for myelin separation. The resultant cell-suspension was then incubated with CD11b microbeads for microglia or with CD45 microbeads for total leucocytes, and the desired population was isolated on MACS MS magnetic columns. Murine microglia were then seeded on poly-L-Lysine (PLL)-coated plates in microglia medium (ScienCell, California, USA).

Primary murine bone marrow-derived macrophages (BMDM) isolation. Bone marrow cells were freshly isolated from the tibias and the femurs of 8-12 weeks old C57BL/6 mice (Envigo CRS, Israel). Bone marrow cells were extracted using 25-gauge syringe and passed through a 70 µm nylon strainer (Corning, Israel). For macrophages differentiation cells were incubated with 50 ng/ml recombinant murine GM-CSF for one week. The medium was replaced 4 days post cells isolation. Cells were tested for CD11b and F4/80 expression using flow cytometry.

Conditioned medium (CM) preparation. To generate CM, GB cells or microglia were seeded in their complete medium. Forty-eight hours post seeding, medium was replaced with starvation medium (0-2% serum) for an additional 24-48 h after which CM was collected.

Tumor spheroids. Tumor spheroids were prepared from mCherry/iRFP/GFP-labeled patient-derived, human U251 or murine GB cells (GL261, iAGR53, PNp53, EGFRviii-shP16), either alone or co-cultured with unlabeled primary human/murine microglia (4:1 ratio), using our modified hanging-drop method, as we previously described [35]. For assessment of cell invasion, GB spheroids (4,000 cells/sphere) were embedded in Matrigel (Corning), seeded in a 96-well plate, and incubated with complete DMEM or complete DMEM supplemented with SELPi (0.5 µM). 3D spheroid invasion was visualized after 24-72 h with an EVOS FL Auto cell imaging system (ThermoFisher Scientific, Massachusetts, USA). For FACS analysis, GB spheroids (8,000 cells/sphere) were embedded in Matrigel, seeded in a 12-well plate, and treated with complete DMEM, 0% serum microglia medium or microglia CM for 24-48 h. Cells were then recovered using Cell Recovery Solution (Corning) and further analyzed by FACS.

Flow cytometry. For flow cytometry assays, cells were harvested using a cell scraper, and were then washed with PBS followed by additional washes with PBS supplemented with 1% BSA and 5 mM EDTA (FACS buffer). Tumor spheroids were recovered from Matrigel using Cell Recovery Solution (Corning) and washed with FACS buffer. To validate the purity of the microglia preparations, CD11b isolated cells or human microglia were incubated with TMEM119 and P2Y12-labeled antibodies for 1 h. For human TMEM119, primary antibody was unlabeled and cells were washed and incubated with secondary, Alexa 488 conjugated antibody. To assess P-Selectin and PSGL-1 expression, GB cells and microglia were seeded alone, co-cultured in a 2D petri dish, or grown as spheroids seeded in Matrigel. Cells were treated with control medium or CM for 48 h. They were then incubated with mouse anti-human P-Selectin antibody for 1 h on ice, and then were washed, and incubated with Alexa-488 labeled anti-mouse IgG binding protein for 1 h on ice. Alternatively, cells were incubated with rat anti-human PSGL antibody, and then were washed, and incubated with goat anti-rat, Alexa-488 labeled antibody for 1 h on ice. For evaluation of CD163, CD206, CD38 and PSGL1 expression by microglia and BMDM, cells were seeded in 2D petri dishes treated with DMEM 0% serum, PD-GB4 CM, PD-GB4 CM + SELP neutralizing antibody (2 µg/ml) or SELPi or rSELP + SELPi for 48 h. Alternatively, human microglia were grown in spheroids containing either iRFP labeled control WT PD-GB4 cells or shSELP PD-GB4 cells. Cells were then incubated with labeled anti- CD163, CD206, CD38 or with unlabeled PSGL-1 antibody for 1 h on ice, before being washed and incubated with Alexa-488 labeled secondary antibody for PSGL-1 expression for 1 h on ice. Microglia from spheroids were identified by negative iRFP staining. For in vitro cytotoxicity of SELPi, U251 and PD-GB4 GB cells were seeded in 6 well plates (1 × 10⁵ cells per well) for 72 h, treated with DMSO (2.5⁻⁴) or with 0.5,1, 2, 5 and 10 µM of SELPi. Following 72 h, cells were trypsinized and staining for annexin-FITC and PI was performed as the manufacturers recommended. For immune-infiltration assessment, CD45 cells freshly isolated from GB tumors were divided into two panels: (1) T cell panel- cells were incubated with FITC-labeled anti-CD3, APC-labeled anti-CD8 and Vio-Blue anti-CD4 antibodies; (2) Treg panel- cells were incubated with Vio-Blue anti-CD4 and Alexa-647 anti-FOXP3. Cells were incubated for 1 h on ice. Single stained cells for each antibody and a pool of the corresponding isotype control were used as negative staining controls. Fluorescence intensity was assessed using either an Attune flow cytometer (Life Technologies) or a Gallios™ flow cytometer (Beckman Coulter, USA) and the results were analyzed by Kaluza software (Beckman Coulter, USA).

Gene knockdown and fluorescence-labeling by retroviral infection. Human embryonic kidney 293T (HEK 293T) cells were co-transfected with the specific expression/knockdown plasmid and the compatible packaging plasmids (pMD.G.VSVG and pGag-pol.gpt). Supernatant containing retroviral particles was collected 48 h post transfection. GB cells or microglia were incubated with the virus for 48 h and positive cells were selected by puromycin resistance for mCherry, shSELP, and shNC infections, and hygromycin for iRFP infection. All retroviral infections showed 70-90% positive infected cells.

Co-culture proliferation assay. Primary murine/human microglia were seeded in a 24 well plate (Corning) at different concentrations, and the same amount of mCherry-labeled murine GL261 (1:4, 1.5:1, 3:1, 6:1 ratios). iRFP-labeled patient-derived or human U251 GB cells were added 24-72 h post seeding (2:1). The plates were then incubated for 96 h (37° C.; 5% CO₂) in microglia medium and proliferation of fluorescently-labeled cells was measured by the IncuCyte Zoom Live cell analysis system (Essen Bioscience). GB cells in microglia medium were used as a control.

Wound healing assay. iRFP-labeled human GB cells, untreated or shRNA-infected, were seeded in a 96-well plate, either alone or with primary human microglia (5:1 ratio) at 100% confluence, and were treated with naive microglia medium (not exposed to cells) or microglia CM. Twenty-four hours post-seeding, a wound maker (Essen Biosciences) was used to create a uniformed wound to the monolayer and then the healing process was monitored and analyzed over the next 48-96 h with the IncuCyte Zoom live-cell analysis system.

Transwell migration assay. Murine GL261, human U251 or PD-GB cells (1 × 10⁵ cells) were seeded in 8 µm inserts (Costar Inc., USA) coated with fibronectin in 0% serum DMEM. After 2 h, once the cells had attached, inserts were transferred to fresh 24-well plates with either microglia medium alone or with the addition of 1 × 10⁵ human or murine microglia seeded in microglia medium. For evaluation of human microglia migration, primary human microglia (1 × 105 cells) were seeded in 8 µm inserts (Costar Inc., USA) coated with fibronectin in 0% serum DMEM. Following cell attachment, inserts were transferred to fresh 24 well plates with either 0% serum DMEM or untreated GB cells seeded in 0% serum DMEM. The cells were allowed to migrate for 5-20 h, before fixation and staining (Hema 3 Stain System; Fisher Diagnostics, USA). The stained migrated cells were imaged using an EVOS FL Auto cell imaging system (ThermoFisher Scientific). The numbers of migrated cells per membrane were evaluated in the captured images by ImageJ software.

Cytokine array. Cytokine secretion following GB-microglia interactions was assessed by culturing murine GL261 GB cells (2 × 10⁴ cells/ml) and primary murine microglia (7 × 10⁴ cells/ml), either alone or together, in microglia medium for 72 h. Medium was then collected, concentrated x20 (Amicon Ultra Centrifugal filters), and analyzed for cytokine secretion by a Cytokine Array (R&D). Cytokine secretion following GB-microglia interactions in human GB model was assessed by culturing human PD-GB4 GB cells (1 × 105 cells/ml) and primary murine microglia (6 × 104 cells/ml), either alone or together, in microglia medium for 72 h. Medium was then collected, concentrated x20 (Amicon Ultra Centrifugal filters), and analyzed for cytokine secretion by a human Cytokine Array (RayBiotech). Cytokines secreted by microglia were detected by incubating primary human/murine microglia (1 × 10⁵ cells/ml) in PLL-coated plates for 24 h before treatment with 0% serum DMEM or 0% serum DMEM supplemented with rSELP (1 µg/ml) for 24 h. The medium was replaced with fresh medium for an additional 24 h before collection and concentration x20 (Amicon Ultra Centrifugal filters). Secreted cytokines were detected by Protein Profiler -Cytokine Array. Membranes were visualized by a myECL imager or the iBright imaging system (ThermoFisher Scientific). Cytokine levels were quantified using ImageJ software.

ELISA. PD-GB cells, U251 cells and primary human microglia (1 × 10⁵ cells/ml) were seeded separately in PLL-coated plates and treated with each other’s CM or naive medium for 24 h. The medium was replaced with fresh medium for an additional 24 h, and was then collected and concentrated (Amicon Ultra Centrifugal filters). SELP levels in the medium were detected by a SELP ELISA kit. Optical density was measured by a SpectraMax M5e multi-detection microplate reader system (Molecular Devices, California, USA).

Nitric oxide secretion evaluation. Primary human/murine microglia (1 × 10⁵ cells/ml) were seeded in PLL-coated plates. Twenty-four hours post-seeding, cells were treated with: 0% serum DMEM, 0% serum DMEM supplemented rSELP (1.5 µg/ml), 0% serum DMEM exposed to rSELP and SELPi (0.5 µM), 0% serum DMEM exposed to rSELP and anti-PSGL-1 neutralizing antibody (2.5 µg/ml), 0% serum DMEM exposed to rSELP and anti-CD44 neutralizing antibody (2.5 µg/ml), or 0% serum DMEM exposed to rSELP and anti-CD24 neutralizing antibody (2.5 µg/ml) for 24 h. Alternatively, macrophages were seeded in 96 well plates (5 × 105 cells/ml) treated with macrophages medium, macrophages medium supplemented with rSELP (3.5 µg/ml), macrophages medium supplemented with rSELP and SELPi (0.5 µM), macrophages medium supplemented with rSELP and anti-PSGL-1 neutralizing antibody (2.5 µg/ml), macrophages medium supplemented with rSELP and anti-CD44 neutralizing antibody (2.5 µg/ml) or macrophages medium supplemented with rSELP and anti-CD24 neutralizing antibody (2.5 µg/ml) for 24 h. Medium was then collected for evaluation of nitrite levels by the Total Nitric Oxide and Nitrate/Nitrite Parameter Assay Kit. Optical density was measured by a SpectraMax M5e multi-detection microplate reader system (Molecular Devices).

Phagocytosis assay. Primary human/murine microglia were seeded in 96 well plates (1 × 105 cells/ml) in microglia medium, microglia medium supplemented with rSELP (1.5 µg/ml for human microglia and 3.5 µg/ml for murine microglia), microglia medium supplemented with rSELP and SELPi (0.5 µM), microglia medium supplemented with rSELP and anti-PSGL-1 neutralizing antibody (2.5 µg/ml), microglia medium 13 supplemented with rSELP and anti-CD44 neutralizing antibody (2.5 µg/ml) or microglia medium supplemented with rSELP and anti-CD24 neutralizing antibody (2.5 µg/ml) for 48 h. The medium was then replaced by fresh microglia medium containing green fluorescence latex beads for 2-6 h and washed with PBS supplemented with Hoechst staining solution for 10 min. For evaluation of murine macrophages phagocytosis abilities, macrophages were seeded in 96 well plates (5 × 105 cells/ml), treated with macrophages medium, macrophages medium supplemented with rSELP (3.5 µg/ml), macrophages medium supplemented with rSELP and SELPi (0.5 µM), macrophages medium supplemented with rSELP and anti-PSGL-1 neutralizing antibody (2.5 µg/ml), macrophages medium supplemented with rSELP and anti-CD44 neutralizing antibody (2.5 µg/ml) or macrophages medium supplemented with rSELP and anti-CD24 neutralizing antibody (2.5 µg/ml) for 48 h. Cells were incubated with latex beads for 3.5 h and washed with PBS supplemented with Hoechst staining solution for 10 min. Phagocytosis was visualized using the EVOS FL Auto cell imaging system (ThermoFisher Scientific) and analyzed by ImageJ software.

Gene expression analysis. SELP mRNA expression was evaluated using qPCR. PD- GB4 cells, U251 cells and primary human microglia (1 × 10⁵ cells/ml) were seeded separately and treated with each other’s CM or naive medium for 48 h.

For qPCR evaluation of SELP silencing, GB cells were seeded in complete DMEM (2 × 10⁵ cells) for 72 h.

To evaluate microglial expression of IL-10 TGF-β, ARG1 and iNOS by primary human microglia (1 × 106 cells), cells were seeded in PLL-coated plates. Twenty-four hours post-seeding cells were treated with: 0% serum DMEM alone, or exposed to rSELP (1.5 µg/ml), or exposed to rSELP and SELPi (0.5 µM), or exposed to rSELP and anti-PSGL-1 neutralizing antibody (2.5 µg/ml), or exposed to rSELP and anti-CD44 neutralizing antibody (2.5 µg/ml), or exposed to rSELP and anti-CD24 neutralizing antibody (2.5 µg/ml) for 48 h.

To evaluate microglial expression of IL-10 and TGF- β by primary murine microglia, cells were freshly isolated and seeded (1 × 10⁶ cells) in PLL-coated plates for 4-6 days. Then, cells were treated with: 2% serum-supplemented microglia medium alone, or exposed to rSELP (3.5 µg/ml), or exposed to rSELP and SELPi (0.5 µM), or exposed to rSELP and anti-PSGL-1 neutralizing antibody (2.5 µg/ml), or exposed to rSELP and anti-CD44 neutralizing antibody (2.5 µg/ml), or exposed to rSELP and anti-CD24 neutralizing antibody (2.5 µg/ml) for 48 h.

RNA isolation. EZ-RNA II total RNA isolation kit (Biological Industries Ltd., Israel) was used to isolate total RNA, according to the manufacturer’s protocol. Briefly, samples were lysed with 0.5 ml Denaturing Solution/10 cm² culture plate. Water saturated phenol was then added, and the samples were centrifuged. Isopropanol was added to precipitate the RNA and the centrifuged RNA pellet was washed with 75% ethanol, centrifuged, and re-suspended with ultra-pure double distilled water. RNA concentration was evaluated using a NanoDrop® ND-1000 Spectrophotometer according to the manufacturer’s V3.5 User’s Manual (Nano-Drop Technologies, Wilmington, DE).

cDNA synthesis. qScript™ cDNA synthesis kit for RT-PCR was used to synthesize cDNA, according to the manufacturer’s protocol. Briefly, 1 µg of total RNA sample was mixed with qScript Reverse Transcriptase, dNTPs, and nuclease free water. The reaction tube was then incubated at 42° C. for 30 min and heated at 85° C. for 5 min to stop cDNA synthesis.

Real-time PCR. The expression level of target genes was assessed by SYBR green real-time PCR (StepOne plus, Life Technologies) and normalized to GAPDH housekeeping gene. Primers used were:

Human SELP: forward- 5′- TCCTTGAGAGCGTTTCAGTATG -′
3 (SEQ ID NO: 1),

reverse -5′ -CTGTCCACTGTCCCAAGTTAT C -′3 (SEQ ID
NO: 2

Murine SELP: forward- 5′- GAC TTTGAGCTACTGGGATCTG
-3′ (SEQ ID NO: 3),

reverse -5′-CAG GAA GTG ATG TTA TGC CTT TG -3′ (S
EQ ID NO: 4)

Human IL-10: forward- 5′- CGCATGTGAACTCCCTGG -3′ (
SEQ ID NO: 5),

reverse- 5′ -TAGATGCCTTTCTCTTGGAGC -3′ (SEQ ID NO
: 6)

Human TGF-β: forward- 5′- GCCTTTCCTGCTTCTCATGG -3′
(SEQ ID NO: 7),

reverse- 5′ -GTACATTGACTTCCGCAAGGA -3′ (SEQ ID NO
: 8)

Human/ murine GAPDH: forward- 5′- ATTCCACCCATGGCAA
ATTC -3′ (SEQ ID NO: 9),

reverse -5′- GGATCTCGCTCCTGGAAGATG -3′ (SEQ ID NO:
10)

Human iNOS: forward- 5′- CGCCTTTGCTCATGACATTG -′3
(SEQ ID NO: 11),

reverse -5′ -TCAAACGTCTCACAGGCTG C -′3 (SEQ ID NO
: 12)

Human ARG1: forward- 5′- AGGTCTGTGGGAAAAGCAAG -′3
(SEQ ID NO: 13),

reverse -5′ -GCCAGAGATTCCAATTG -′3 (SEQ ID NO: 14
)

Murine IL-10: forward- 5′- TGAATTCCCTGGGTGAGAAGC -
3′ (SEQ ID NO: 17),

reverse- 5′ -CACCTTGGTCTTGGAGCTTATT -3′ (SEQ ID N
O: 15)

Murine TGF-β: forward- 5′- ACTGGAGTTGTACGGCAGTG -3
′ (SEQ ID NO: 18),

reverse- 5′ -GGGGCTGATCCCGTTGATT -3′ (SEQ ID NO:
16)

Western Blot. To assess the activation of Nf-kb pathway in the presence of rSELP, human microglia were exposed for 2 h to serum-free medium followed by 3 h incubation with serum-free medium supplemented with rSELP (1 µg/ml). Cells were subsequently lysate in RIPA buffer supplemented with fresh proteases and phosphatases inhibitors (Invitrogen, USA). Cells lysate were loaded into a 10% acrylamide/bis-acrylamide gel and the proteins were transferred on nitrocellulose filter and blocked with 5% BSA in Tris-HCl buffer with 1 % Tween. Phosphorylated p65 (p-p65) and total p65 antibodies were incubated with the nitrocellulose filter for overnight at 4° C. Vinculin antibody was incubated for 1h at RT and used as housekeeping to normalize the expression of p-p65 and p65. Rabbit secondary antibody HRP conjugated was incubated for 1 h at RT. SuperSignal™ West Pico Plus chemiluminescent substrate (Thermo Scientific) was added to the membrane, and images were developed using iBright 1500 instrument (Life Technologies, USA). Pixel density of the corresponding protein bands was quantified using ImageJ software.

Animal models. In order to assess microglia density and SELP expression in GB tumors by histology, 6 week-old male C57BL/6 mice (Envigo CRS, Israel) were anesthetized by ketamine (150 mg/kg) and xylazine (12 mg/kg) injected intraperitoneally (IP), and mCherry-labeled murine GL261 GB cells (5 × 10⁴ cells) were stereotactically implanted into their striatum (N = 10). Patient-derived and iRFP-labeled U251 human GB cells (5 × 10⁴ cells) were stereotactically implanted into the striatum of 6 week-old male SCID mice (Envigo CRS, Israel) (N = 10). Tumor development was followed by MRI (MR Solutions) and CRI Maestro™ imaging system.

To investigate the effect of SELP shRNA on tumor growth in the murine GB model, control wild-type (WT), shSELP, or shNC GL261 GB cells (5 × 10⁴ cells) were stereotactically implanted into the striatum of 6 week-old male C57BL/6 mice (N=15) (Envigo CRS, Israel). Tumor volume and development were followed by MRI (MR solutions). Mice were euthanized when they lost 10% body weight in a week, had lost 20% of their initial weight, or when neurological symptoms appeared. Six mice per group were euthanized at day 17 post injection and the brains were resected for further analysis by immunostaining, flow cytometry, and evaluation of gene expression.

Single-cell RNA-seq of CD11b-positive cells and additional flow cytometry analysis was performed following stereotactical implantation of 5 × 10⁴ control - WT, shSELP or shNC GL261 GB cells into the striatum of 6 week-old male C57BL/6 mice (N = 6) (Envigo CRS, Israel). Tumor volume and development were followed by MRI (MR solutions). Mice were euthanized at day 17 post injection and the brains were resected. A pool of 3 brains per group of mice was used to generate cell suspensions as described above, and CD11b microbeads were used to isolate microglia/macrophages for single-cell RNA-seq analysis. The remaining cell-suspension was treated with CD45 microbeads and flow cytometry analysis was performed as described above. Alternatively, CD11b and the remaining CD45-isolated cells, as well as the rest of the cell-suspension, were mixed at equal amounts and analyzed for whole-tumor single-cell RNA-seq.

To assess the effect of SELP shRNA on tumor growth in the human GB model, control WT, shSELP or shNC human iRFP-labeled U251 or GB-PD4 cells (5 × 10⁴ cells) were stereotactically implanted into the striatum of 6 week-old male SCID mice (N = 9) (Envigo CRS, Israel). Tumor volume and development were followed by MRI (MR solutions). Mice were euthanized when they lost 10% body weight in a week, had lost 20% of their initial weight, or when neurological symptoms appeared. Three mice per group were euthanized at day 27 for U251 or day 35 for PD-GBpost injection and brains were resected for further analysis by immunostaining.

To examine the effect of SELPi treatment in the human U251 GB model, iRFP-labeled U251 cells (5 × 10⁴ cells) were stereotactically implanted into the striatum of 7 week-old male SCID mice (Envigo CRS, Israel) (N = 10). SELPi (0.8 mg/ml) was dissolved in DMSO 0.01%), polyethyleneglycol (93.2 mg/ml), Tween-80 (14.8 mg/ml), and DDW. Mice were treated intravenously (IV) twice a week with PBS, 16 mg/kg SELPi or vehicle (0.01% DMSO, 93.2 mg/ml polyethyleneglycol and 14.8 mg/ml Tween-80 in DDW). Tumor development was followed by monitoring the fluorescence intensity using the CRI Maestro™ imaging system. Mice were euthanized at day 26 post injection and brains were resected for further analysis by immunostaining.

To examine the effect of SELPi treatment in the murine GL261 GB model, GL261 cells (5 × 10⁴ cells) were stereotactically implanted into the striatum of 7 week-old male C57BL/6 mice (Envigo CRS, Israel) (N = 5) and treated with SELPi three times a week. Tumor volume was evaluated by MRI. Mice were euthanized when they lost 10% body weight in a week, had lost 20% of their initial weight, or when neurological symptoms appeared. Mice were euthanized at day 17 post injection and brains were resected for further analysis by immunostaining.

To examine the effect of SELPi treatment in the murine mesenchymal iAGR GB model, GFP-labeled iAGR cells (1 × 10⁵ cells) were stereotactically implanted into the striatum of 7 weeks-old male C57BL/6 mice (Envigo CRS, Israel) (N = 15) and treated with 16 mg/kg SELPi, IV, QOD. Tumor volume was evaluated by MRI. Mice were euthanized when they lost 10% body weight in a week, had lost 20% of their initial weight, or when neurological symptoms appeared. Twelve days post cell inoculation, 3 mice per group were euthanized and brain were resected and further processed for histology analysis. To examine the effect of SELPi treatment in the PD-GB model, 5 × 10⁴ iRFP-labeled patient-derived cells were stereotactically implanted into the striatum of 7 week-old male SCID mice (Envigo CRS, Israel) (N = 5). Brain cannulas (NBT) were implanted intraventricularly in the co-lateral hemisphere and the mice were treated stereotactically via the cannulas (1 µl at 0.2 µl/min flow rate) with PBS, 2 mg/kg SELPi dissolved in DMSO or vehicle (DMSO). Three weeks post cell-inoculation, the mice were euthanized, the cannulas were removed, and tumor volume was evaluated by MRI (MR solutions). Brains were resected for immunostaining.

To test the effect of SELPi treatment in vivo on mice blood chemistry and blood count, c57/BL mice were injected with 16 mg/kg SELPi IV. Twenty-four hours following injecting, mice were euthanized and blood was collected. Blood samples were analyzed by AML Ltd (Herzliya, Israel). For all models, 50 × 10³ GB cells were stereotactically injected in 5 µl at 1 µl/min flow rate. Injection coordination relative to the mouse Bregma point were: 2 mm lateral (left), 0.5 mm anterior, 3.5 mm ventral.

SELPi treatments. KF 38789 (SELPi) is a commercially-available P-Selectin inhibitor manufactured by Tocris BioScience. SELPi was shown to be a selective P-Selectin inhibitor (IC50 = 1.97 µM) with no inhibiting effects on L-Selectin or E-Selectin. It was shown to block SELP-binding in vitro and in vivo. This compound is commonly used for research of immune-related processes such as tumor metastases formation. For the experiment described above, SELPi (0.8 mg/ml) was dissolved in DMSO (0.01%), polyethyleneglycol (93.2 mg/ml), Tween-80 (14.8 mg/ml) and DDW. Mice were injected with 16 mg/kg IV, twice a week, three times a week or QOD as described above. As control (vehicle), mice were injected with DMSO (0.01%), polyethyleneglycol (93.2 mg/ml), Tween-80 (14.8 mg/ml), in DDW at equivalent volume.

Droplet-based single-cell RNA-Seq. GL261 GB tumor cells were initially exposed to shSELP or control shRNA plasmid as described above, and injected into mice. Microglia/macrophages (CD11b+), T cells (CD11b-CD45+) and tumor cells (the remaining cell suspension) isolated from the tumors were mixed in equal amounts (see animal model part). We also isolated Microglia/Macrophage cells (CD11b+) only from the two groups of GL261 GB tumor-bearing mice. Cells were encapsulated into droplets, and libraries were prepared using Chromium Single Cell 30 Reagent Kits v3 according to manufacturer’s protocol (10X Genomics). The generated single cell RNA-seq libraries were sequenced using a 75 cycle NextSeq 500 high output V2 kit.

Droplet-based single-cell RNA-Seq data processing. Gene counts were obtained by aligning reads to the mm10 genome using CellRanger software (v1.3 10X Genomics). To remove doublets and poor-quality cells, we excluded cells that contained more than 10% mitochondrially-derived transcripts, or where less than 500 genes were detected. Among the retained cells, we considered only genes present in > 3 cells, which yielded for the global experiment 4,654 and 5,708 cells from mice exposed to shSELP or control tumors respectively. We then randomly selected 4,580 from each group. For the microglia/macrophages focused experiment 4,409 and 3,376 cells from mice exposed to shSELP or control tumors respectively. We then randomly selected 3,300 from each group. Transcript counts for each library were scaled using the ScaleData function in Seurat, which scales and centers features in the dataset. The results were then normalized using the LogNormalize function in Seurat, by which the feature counts for each cell are divided by the total counts for that cell and multiplied by the scale factor. This is then natural-log transformed using loglp. For principal component analysis (PCA) and clustering, we used a log-transformed expression matrix. The top 12 and 8 PCs, from the global and microglia/macrophages focused experiment respectively, were selected for subsequent tSNE analysis, determined by a drop in the proportion of variance explained by subsequent PCs. We confirmed that the resulting analyses were not particularly sensitive to this choice.

Single cell RNA-seq clustering. The FindClusters function in Seurat was used to identify clusters of cells by a shared nearest neighbor (SNN) modularity optimization-based clustering algorithm. First, we calculated the k-nearest neighbors and constructed the SNN graph. We then optimized the modularity function to determine clusters as previously described [41]. Shifts in the distribution of cells from mice exposed to shSELP or control tumors for each of the clusters were calculated using Fisher’s exact test. To further confirm the annotation of the tumors clusters we used inferred CNV analysis implementation of the R package InferCNV (www(dot)github(dot)com/broadinstitute/inferCNV). We first run the analysis on the cell clusters we identified as tumor cells (with a separation to shSELP and shNC). Next, we used the rest of the cell clusters as a reference. Thus, we were also been able to confirm that we did not miss any other tumor cluster. The analysis was run with the following arguments: “denoise”, default hidden markov model (HMM) settings, “cutoff” of 0.1 as suited for 10x data - set, and a mouse gene map originated in the Mouse Genome Informatics data base.

Genes differentially expressed between clusters. The FindAllMarkers function in Seurat was used to find marker genes that were differentially expressed between clusters. This function identifies differentially expressed genes between two groups of cells using a Wilcoxon Rank Sum test with limit testing chosen to detect genes that display an average of at least 0.25-fold difference (log-scale) between the two groups of cells and genes that are detected in a minimum fraction of 0.25 cells in either of the two populations. This step was intended to speed up the function by not testing genes that are very infrequently expressed.

Visualization of single cell data. To generate tSNE plots [42, 43] of single cell profiles, the scores along the 8 significant PCs described above were used as input for the R implementation of tSNE, by the RunTSNE function in Seurat. Heatmaps were generated using DoHeatmap function in Seurat for the top 10 DE genes in each cluster.

Single-cell gene signature scoring. Single-cell gene signature scoring was done as described previously [44]. Briefly, as an initial step, the data was scaled (z-score across each gene) to remove bias towards highly expressed genes. Given a gene signature (list of genes), a cell-specific signature score was computed by first sorting the normalized scaled gene expression values for each cell followed by summing up the indices (ranks) of the signature genes. For gene-signatures including both up-regulated and down-regulated genes, two ranking scores were obtained separately, and the down-regulated associated signature score was subtracted from the up-regulated generated signature score. A contour plot which takes into account only those cells that have a signature score above the indicated threshold was added on top of the tSNE space, in order to further emphasize the region of highly scored cells.

Given a gene signature (list of genes), a cell-specific signature score was computed by averaging the score of two methods. In the first method [44], as an initial step, the data was scaled (z-score across each gene) to remove bias towards highly expressed genes. Given a gene signature (list of genes), a cell-specific signature score was computed by first sorting the normalized scaled gene expression values for each cell followed by summing up the indices (ranks) of the signature genes. For gene-signatures including both up-regulated and down-regulated genes, two ranking scores were obtained separately, and the down-regulated associated signature score was subtracted from the up-regulated generated signature score. In the second method, total expression of the up-regulated genes was summed and subtracted from the total expression of the down-regulated. Finally, we scaled and averaged the two scoring values.

MRI imaging. Tumor bearing mice were imaged at the Sackler Cellular & Molecular Imaging Center (SCMIC), Tel Aviv University. Mice were anesthetized by ketamine (150 mg/kg) and xylazine (12 mg/kg) injected IP. T1 with contrast agent (Magnetol, Gd-DTPA, Soreq M.R.C. Israel Radiopharmaceuticals) and T2 weighted images were taken by 4.7T MRI - MRS 4000™ (MR solutions).

Intravital imaging. The CRI Maestro™ non-invasive fluorescence imaging system was used to follow tumor progression in mice bearing mCherry- or iRFP- labeled U251 tumors. Mice were anesthetized by ketamine (150 mg/kg) and xylazine (12 mg/kg) injected IP, and were placed inside the imaging system. Multispectral image-cubes were used through 550-800 nm spectral range in 10 nm steps using excitation (575-605 nm) and emission (645 nm longpass) filter set. Mice autofluorescence and background signals were eliminated by spectral analysis and by applying a linear unmixing algorithm.

Human FFPE specimens. FFPE GB samples were obtained from Tel Aviv Sourasky Medical Center. A total of 60 samples were collected: 36 samples of patients who survived short-term- STS (69% men; 65 ± 2 years; survival 3.7 ± 0.2 months), and 24 samples of patients who survived long-term- LTS (58% men; 56 ± 3 years; survival 48 ± 3.9 months). Normal human brain FFPE samples were obtained from the Lieber Institute (Baltimore, MD, USA).

Frozen OCT tissue fixation. Tumor bearing mice were anesthetized with an IP injection of ketamine (100 mg/kg) and xylazine (12 mg/kg) and perfused with PBS followed by 4% Paraformaldehyde (PFA). Brains were harvested, and incubated with 4% PFA for 4 h, followed by 0.5 M of sucrose (BioLab) for 1 h, and 1 M sucrose overnight (ON). The brains were then embedded in optimal cutting temperature (OCT) compound (Scigen) on dry ice and stored at -80° C.

Immunostaining. OCT embedded tumor samples were cut into 5 µm thick sections. Staining was performed using BOND RX autostainer (Leica). Sections were stained by hematoxylin and eosin (H&E) and immunostained for: proliferating cells using rabbit anti-human/mouse KI67 antibody and Alexa Fluor 488-goat anti-rabbit secondary antibody; Blood vessels using rat anti-mouse CD31 antibody and Alexa Fluor 488-goat anti-rat secondary antibody; IBA1 activated microglia using rabbit anti-mouse IBA1 antibody and Alexa Fluor 488-goat anti-rabbit secondary antibody; CD8-positive T-cells using rat anti-mouse CD8 antibody and Alexa Fluor 488-goat anti-rat secondary antibody; CD4-positive T-cells using rat anti-mouse CD8 antibody and Alexa Fluor 488-goat anti-rat secondary antibody; and FOXP3-positive T-cells using rabbit anti- mouse/human FOXP3 and Alexa Fluor 488-goat anti-rabbit secondary antibody. Prior to antibody incubation, slides were incubated with 10% goat serum in PBS x1 + 0.02% Tween-20, for 30 min to block nonspecific binding sites. Slides were incubated with primary antibodies for 1 h, and then washed and incubated with secondary antibodies for an additional 1 h. They were then washed and treated with ProLong® Gold mounting with DAPI before being covered with coverslips.Patient FFPE samples were stained for Iba1 and human SELP as described above. Stained samples were imaged using the EVOS FL Auto cell imaging system (ThermoFisher Scientific). At least three fields of each individual sample were imaged and quantified using ImageJ software. Quantification of positive staining was performed by measuring the total area stained in each image following background subtraction, using single color images representing the correlated marker.

Statistical analysis. Data are expressed as mean ± standard deviation (s.d.) for in vitro assays or ± standard error of the mean (s.e.m.) for in vivo assays. Statistical significance was determined using an unpaired t-test. p < 0.05 was considered statistically significant. All statistical tests were two-sided. For Kaplan-Meier survival curves, p values were determined using log rank test. For in vivo tumor growth curves, p values were determined using one-way ANOVA, Dunn’s test, or Holm-Sidak’s test.

Data and software availability. The sequence data generated in this study have been deposited in the Gene Expression Omnibus (GEO) and the accession code will be provided prior to publication. The software used for the analyses of each of the data platforms, and integrated analyses are described and referenced in the individual Method Details subsections.

Molecular characterization of human GB samples. The molecular subtypes of our GB FFPE samples from short-term survivals (STS) included at least one proneural, one mesenchymal and one classical subtypes. Human U251 GB cell line is considered as proneural [46]. The patient-derived cell lines exploited in this study are all IDH WT while PD-GB1, PD-GB2 and PD-GB4 are p53-mutated, PD-GB3 is p53 WT and ATRX mutated, and PD-GB4 is ATRX WT.

Molecular characterization of murine GB samples. GL261 is considered to be a mesenchymal-like phenotype [47]. GB cell lines isolated from lenti-induced GB mouse models include: iAGR53 - primary astrocytes from GFAP-Cre mice transduced with lenti-HRasV12-shp53 - Mesenchymal subtype; EGFRviii-shp16- Classical subtype; and PNp53 - derived from a tumor induced in GFAP-Cre mouse (cell of origin neural stem cell, NSC, in the sub ventricular zone) injected with lenti-PDGFB-shp53 - Proneural subtype [39, 40].

### RESULTS

### GB-Microglia Interactions Promote GB Cell Migration and Proliferation

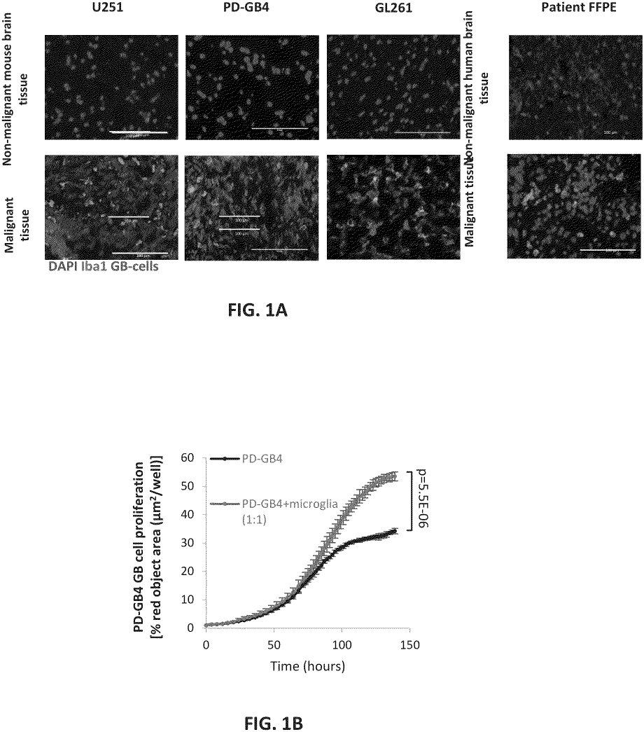

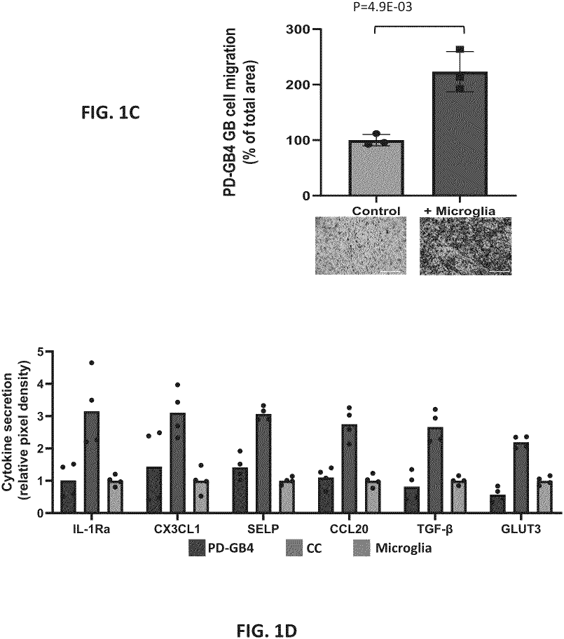

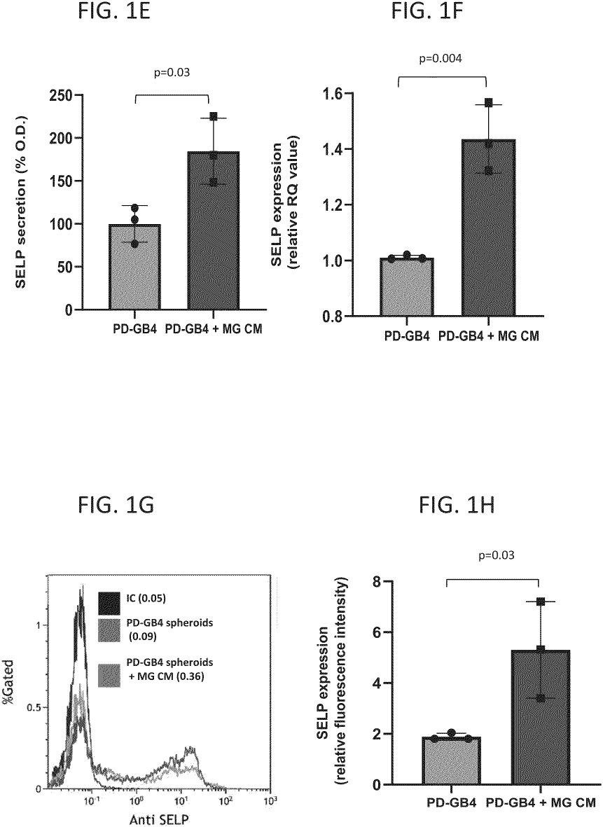

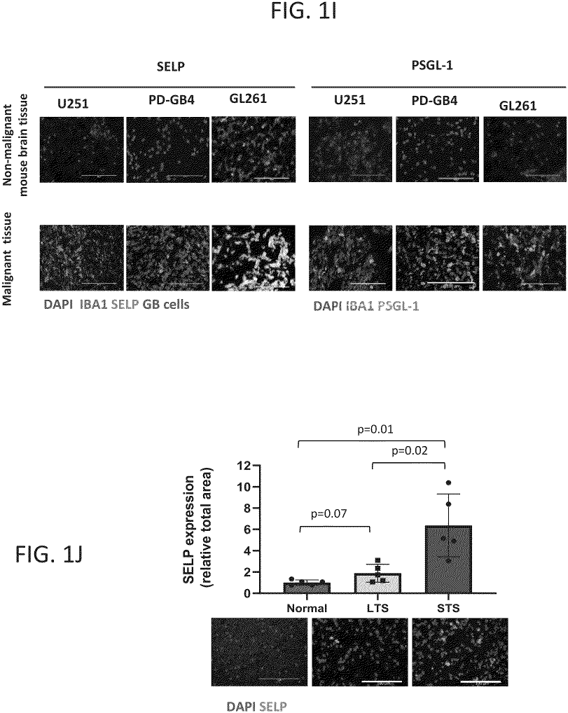

Our initial objective was to investigate the role of microglia in GB progression. Using patient GB samples, and human xenogeneic and murine syngeneic GB mouse models, we detected the presence of activated microglia in the tumor by Iba1 immunostaining (FIG. 1A). We then proceeded to evaluate the effect of microglia on GB cell proliferation and migration in vitro. To that end, we have used commercially available human microglia and isolated murine microglia from naive mice by CD11b positive selection. These isolated microglia were characterized by the specific microglia markers TMEM119 and P2Y12.

Co-culture proliferation assay and TransWell migration assay revealed increased proliferation and migration of patient-derived GB cells (PD-GB4) in the presence of human microglia (FIGS. 1B-C). In addition, we found that PD-GB4 cells facilitate the proliferation and migration of human microglia as well, indicating the reciprocal activation of microglia following the interaction with GB cells in our models. This suggests that GB cells may induce microgliosis in the brain as a result of the neuroinflammation induced by their crosstalk. We observed a direct correlation between the increased proliferation rate of murine GL261 GB cells and the concentration of murine microglia in the culture, and increased migration of GL261 cells towards murine microglia. These findings were reproduced using human U251 GB cell line showing enhanced proliferation and migration of U251 cells following the interactions with human microglia.

### GB-Microglia Interactions Induce High Expression of SELP that Facilitates GB Progression

In order to detect secreted factors that play a key role in GB-microglia interactions and regulation of tumor progression, we performed protein-arrays of human PD-GB4 GB cells and primary human microglia. Our findings revealed several factors were over-secreted following co-culture of GB cells with microglia (FIG. 1D).

We then screened for over-secreted factors in a co-culture of primary murine microglia and murine GL261 GB cells, to verify whether the detected factors are relevant for both human and murine GB models.

Interestingly, in both assays, we found a significant increase in the secretion of SELP in the co-culture medium compared to the mono-cultures, which led us to further investigate the role of SELP in GB progression.

When we used ELISA and RT-PCR to validate the secretion and expression of SELP by human GB cells (PD-GB4), the results revealed enhanced secretion and high expression of SELP following incubation of GB cells with human microglia CM (FIGS. 1F, 1G). As the expression of membrane-bound SELP was low in GB cells grown in traditional 2D cultures (data not shown), we chose to use an in vitro 3D model involving a modified “Hanging-Drop” method that we previously developed to create GB spheroids and seed them in Matrigel [35]. SELP was found to be expressed by PD-GB4 and U251 tumor spheroids, and its level of expression was increased when the spheroids were treated with microglia CM, as demonstrated by flow cytometry (FIGS. 1H, 1I). Furthermore, the results with two patient-derived xenografts (PDX) and GL261 mouse models, revealed positive staining of SELP and PSGL-1 in tumor areas enriched with activated microglia (FIG. 1I).

This was performed using two additional PDX mouse models. We note that SELP staining correlated mainly with the labeled GB cells while PSGL-1 staining seemed to correlate with Iba1 staining. Furthermore, we co-stained for Ki-67 together with Iba1 to validate in vivo the effects of microglia on GB cell proliferation observed in vitro, and found high expression of Ki-67 in areas enriched with Iba1. As SELP is known to be expressed by activated tumor endothelial cells, we co-stained for SELP and CD31 in two human and one murine GB mouse models. As expected, we also found positive staining of SELP on the tumor blood vessels. Since we used anti-mouse SELP antibody, human xenografts showed staining which correlates only with CD31 staining. This is in contrast to the murine model which showed positive staining that correlated with both blood vessels and tumor cells. These findings together with the staining presented in FIG. 1I indicate that both the tumor cells and their associated endothelial cells express SELP in vivo. In order to further validate the expression and clinical relevance of SELP, we obtained FFPE samples from short-term GB survivors (STS, < two months) and long-term GB survivors (LTS, > five years), as well as normal, healthy human brain tissues. Immunostaining of these samples, demonstrated higher expression of SELP in STS tumor samples compared to LTS tumors or normal brain samples (FIG. 1J).

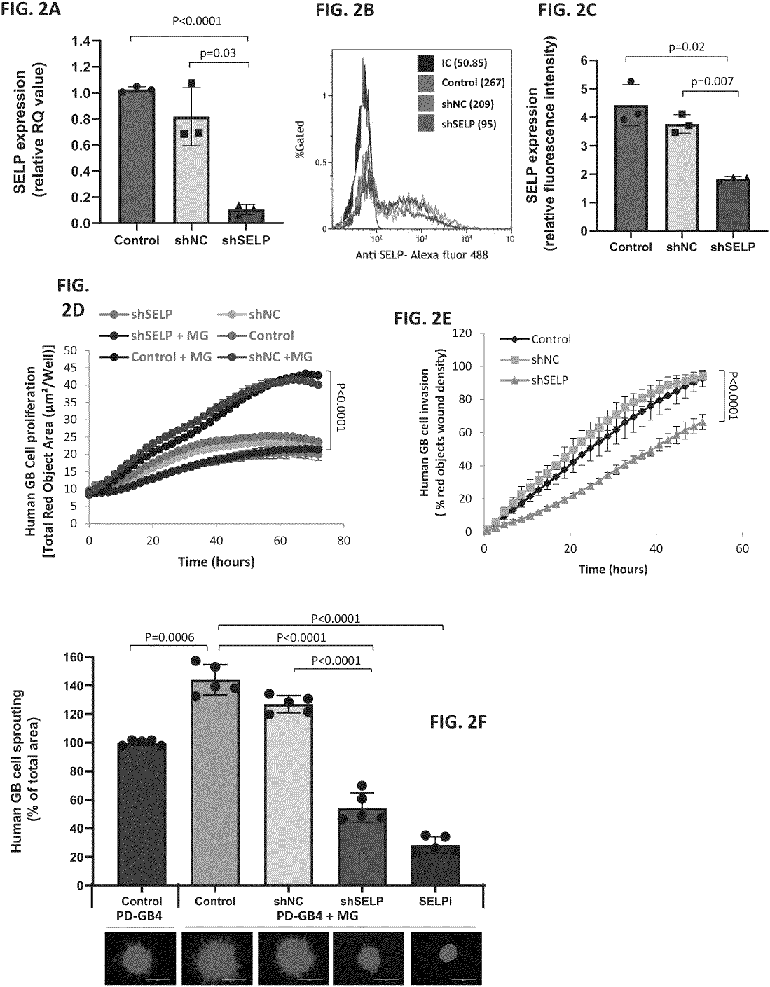

The observation that SELP is expressed by GB cells in vitro and in vivo, and overexpressed in the presence of microglia, prompted us to further investigate the functional role of SELP in GB progression, and specifically in GB cell-microglia (tumor-host) interactions. To that end, we established SELP knockdown GB cells using retroviral infection of SELP-shRNA (FIGS. 2A-C). We mono-cultured or co-cultured GB cells with microglia and followed GB cell proliferation and migration in 2D in vitro models. SELP knockdown GB cells (shSELP) exhibited reduced proliferation and migration compared to negative control shRNA infected (shNC) and control WT cells, in the presence of microglia (FIGS. 2D, 2E). Interestingly, in the absence of microglia, these differences in proliferation were significantly reduced (FIG. 2D), indicating the relevance of SELP in GB-microglia interactions.

The results show that shSELP GB cell proliferation in a co-culture is similar (no significant difference) to that of the WT cell proliferation in mono-culture, and that shSELP cell proliferation in the co-culture is similar to shSELP proliferation in mono-culture. This suggests that SELP knockdown eliminates the differences observed between mono-culture and co-cultures (FIG. 1B), hence inhibiting the microglia-enhanced GB cell proliferation. Since we observed high expression of SELP in our 3D model (FIGS. 1G, H), we co-cultured GB cells and microglia in 3D spheroids. While the invasion of GB cells into the Matrigel was increased in the presence of microglia, SELP knockdown significantly inhibited PD-GB4 3D spheroid invasion (FIG. 2F). To confirm that this effect was due to SELP knockdown, we treated GB-microglia spheroids with a SELP specific small-molecule inhibitor (SELPi). SELPi treatment of GB cells in the presence of microglia resulted in a similar reduction in cell invasion (FIG. 2F), indicating that SELP is an important mediator of GB cell invasion. Inhibition of spheroid invasion following SELP knockdown was also observed using human U251 GB cells, and the effect of SELPi on GB spheroids was observed using human U251, PD-GB 1, PD-GB3, and murine GL.261 GB models. In order to evaluate whether the observed effects of SELPi are not due to cytotoxicity, we have stained the treated cells for apoptosis-necrosis. The results showed slight increase in the percentage of apoptotic cells only when GB cells were treated with high concentrations of 5 and 10 µM SELPi, while no toxicity was observed using 0.5 µM which is the working concentration used for all in vitro experiments. Thus, showing that SELPi delayed the proliferation and invasion of GB cells in the presence of microglia and did not induce direct cell-killing. Since GBs are highly heterogeneous tumors, consisting of several molecular subtypes, we used three lenti-induced murine GB cells which represent the mesenchymal, proneural and classical GB subtypes. We found that the mesenchymal, proneural and classical GB spheroids express high levels of SELP compared to 2D cultures and even higher levels when co-cultured with murine microglia. Moreover, treatment with SELPi significantly reduced the invasion of GB spheroids co-cultured with murine microglia using all three GB subtypes The results showed that SELF mediates GB cell proliferation and invasion in the presence of microglia in 2D and 3D in vitro models using several human and murine GB models.

### GB Cells Promote the M2-Like Anti-Inflammatory/Pro-Tumorigenic Function Of Microglia/Macrophages via SELP-PSGL-1 Axis

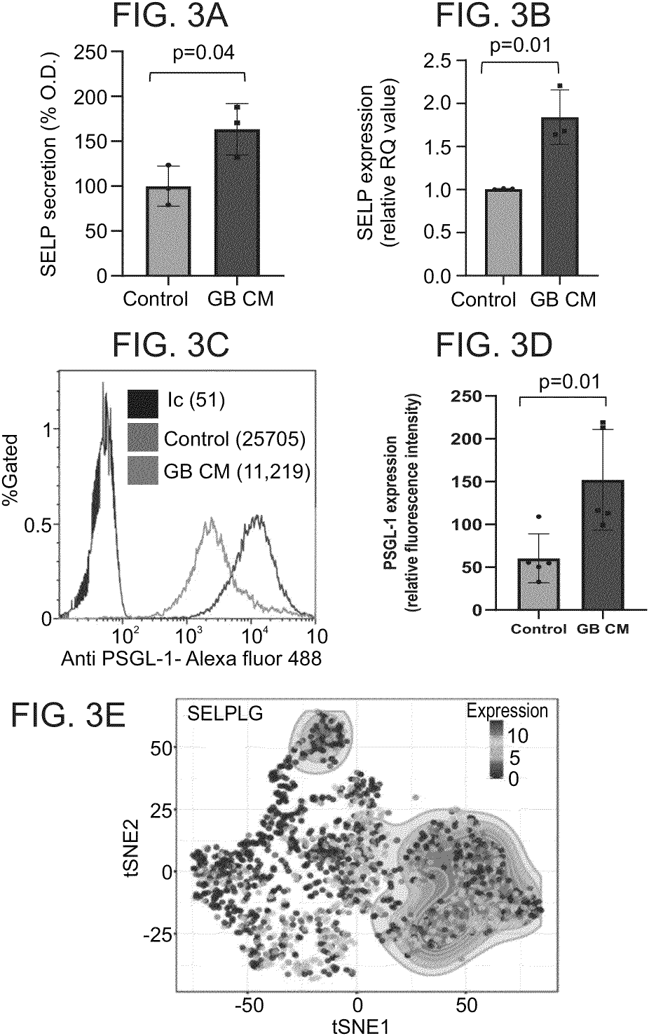

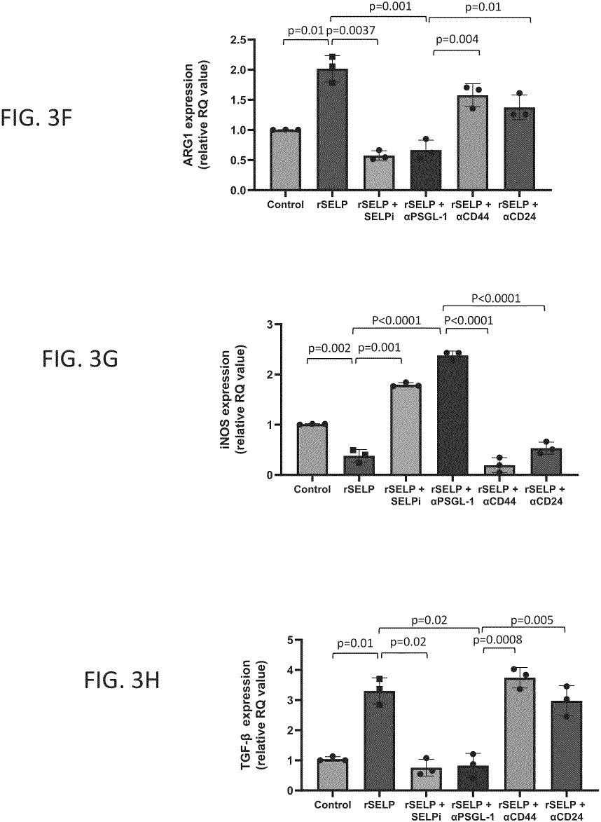

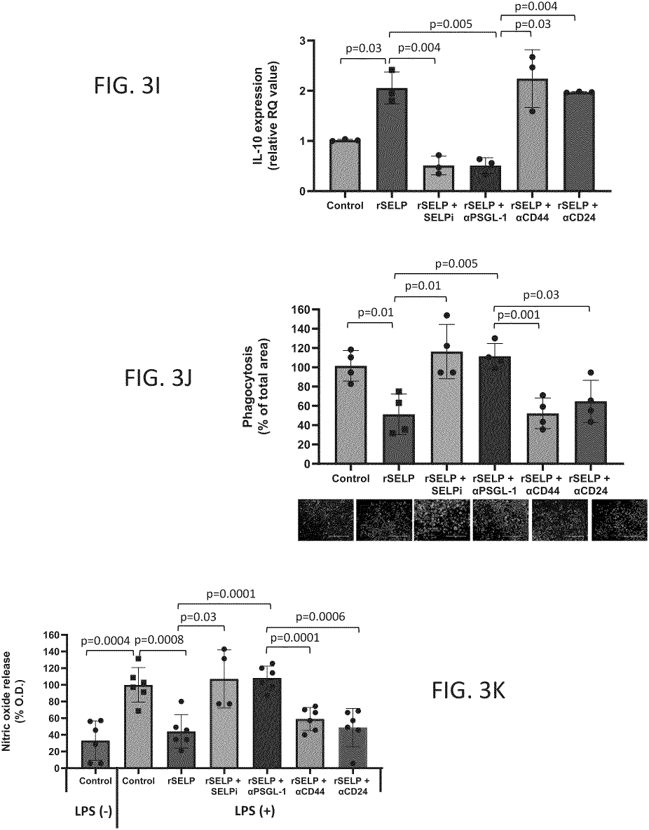

When we investigated the effect of SELP on microglia, we found that not only GB cells, but also microglia, express SELP-mRNA and secrete SELP. Both expression and secretion of SELP were elevated when human microglia were treated with GB CM (FIGS. 3A, 3B). Moreover, microglia upregulated the expression of PSGL-1 when exposed to GB CM (FIGS. 3C, 3D). Indeed, further analysis of single-cell RNA-seq data, obtained by Darmanis et al., of microglia isolated from fresh patient-derived GB samples [48] revealed high expression of PSGL-1 in a population defined as resident microglia (FIG. 3E).

To evaluate the effect of SELP on microglia phenotype, we followed the expression of arginase 1 (ARG1) which is related to immunosuppressive phenotype of tumor-associated macrophages (TAMs). Furthermore, we evaluated the expression of inducible nitric oxide synthase (iNOS) which is involved in nitric oxide production and is associated with pro-inflammatory phenotype, in addition to the assessment of the immunosuppressive cytokines IL-10 and TGF-β (FIGS. 3F-I). We found that treatment with recombinant SELP (rSELP) resulted in increased expression of ARG-1, IL-10 and TGF-β and reduced expression of iNOS. We were able to rescue the effects of rSELP by adding SELPi or anti-PSGL-1 neutralizing antibody on top of rSELP treatment. Interestingly, only partial effect, if any, was observed when neutralizing antibodies against the additional SELP ligands CD44 and CD24 were added (FIGS. 3F-I). Phagocytosis and NO release are functional characteristics of microglia/macrophages associated with pro-inflammatory activation state. A phagocytosis assay with fluorescently-labeled latex beads, demonstrated a reduction in phagocytic activity of human microglia when treated with rSELP (FIG. 3J). Adding SELPi or anti-PSGL-1 neutralizing antibody restored the phagocytic function of human microglia while neutralizing antibodies for CD44 or CD24 did not affect this ability. Finally, we were also able to show that treatment with rSELP reduced the amount of NO released by human microglia following LPS induction. NO levels were restored when we added SELPi or anti-PSGL-1 neutralizing antibody but not when adding anti-CD44 or CD24 neutralizing antibodies (FIG. 3K). These results suggest that SELP mediates the anti-inflammatory phenotype of microglia by binding PSGL-1 and not other SELP ligands. The secretion of IL-10 and TGF-β was also evaluated in the protein level using cytokine array, showing higher secretion by human microglia when treated with rSELP. The cell surface proteins CD163 and CD206 (MRC1), are associated with anti-inflammatory function of microglia/macrophages. Thus, we evaluated their expression by human microglia using flow cytometry. In the presence of PD-GB4 CM, human microglia expressed higher levels of CD163 than untreated cells. This CD163 expression was reduced when microglia cells were treated with PD-GB4 CM supplemented with SELP neutralizing antibody. Treating human microglia with PD-GB4 CM and rSELP induced the expression of CD206 by human microglia while adding SELPi to PD-GB44 CM reduced CD206 expression. Accordingly, microglia isolated from control WT GB-microglia spheroids expressed higher levels of CD163 than microglia isolated from spheroids prepared with shSELP PD-GB4 cells. To reveal the downstream effects of SELP-PSGL-1 axis in microglia, we have explored the activation of the NF-kB pathway. We found an increase in the phosphorylation of the NF-kB protein p65 when human microglia were treated with rSELP compared to untreated cells. Validating the effects of SELP on microglia immunophenotype, we used freshly-isolated primary murine microglia in addition to human microglia. We showed that SELP-PSGL-1 axis mediates the expression of IL-10 and TGF-P by murine microglia and that treatment with rSELP increased the secretion of IL-10 and reduced the phagocytic ability of murine microglia. In order to elucidate whether these findings are restricted to GB-microglia interactions, or apply also to GB interactions with macrophages, we used freshly-isolated bone marrow derived macrophages (BMDM). First, we found that BMDM also express PSGL-1. This expression was elevated when the cells were treated with rSELP and GB CM, and reduced when SELPi was added to GB CM. Then, we observed increased CD206 expression and decreased CD38 expression when BMDM were treated with rSELP. CD38 is known to induce pro-inflammatory cytokines secretion by TAMs. Following the addition of SELPi to rSELP containing medium, the expression of CD206 was reduced while the expression of CD38 was elevated. In addition, we found that SELP-PSGL-1 interactions mediate BMDM phagocytosis activity and nitric oxide release. These results show that SELP-PSGL-1 axis is involved in GB-macrophages interactions as well. The association between PSGL-1 and microglia/macrophages was also explored using the GBM datasets in TCGA. The results of the analysis showed a positive correlation between the expression of microglia gene signature-Tmem119, Cx3cr1, P2ry12, Arg1, Olfm13, Gpr34, Cc14 and Cc13, and PSGL-1 expression in GB patients. Interestingly, we also found a positive correlation between PSGL-1 and CD4; and, following analysis of single-cell RNA-seq data obtained from the Single Cell Portal, we found that PSGL-1 was expressed not only by microglia/macrophages, but also by infiltrating T cells in GB samples. This suggests that the SELP-PSGL-1 axis might also play a role in the interactions with infiltrating T cells.

Taken together, these results demonstrate the immunosuppressive effect of SELF on microglia/macrophages which is mediated by PSGL-1 in contrast to other SELP ligands.

### SELP Inhibition Delays Tumor Growth and Affects the Microenvironmental Landscape in GB Mouse Models

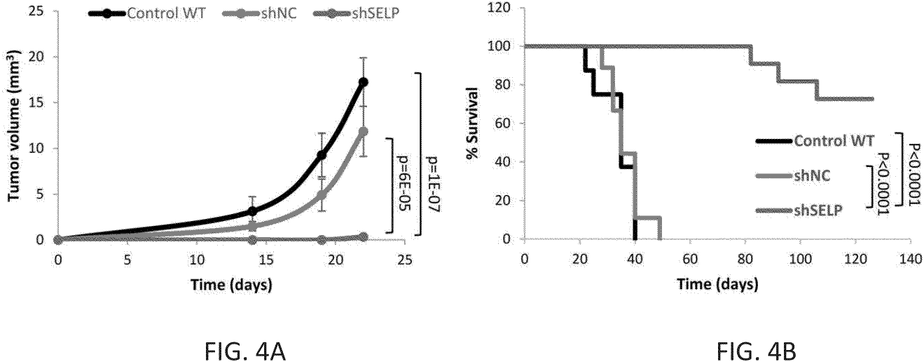

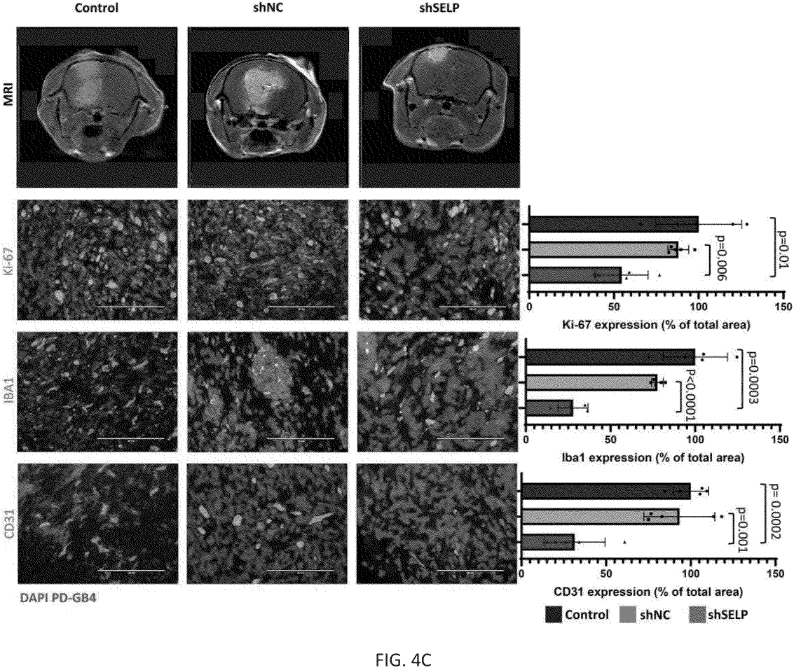

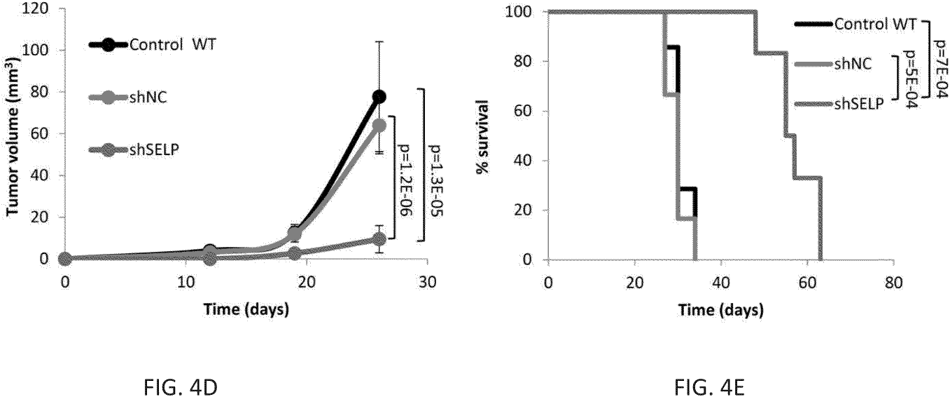

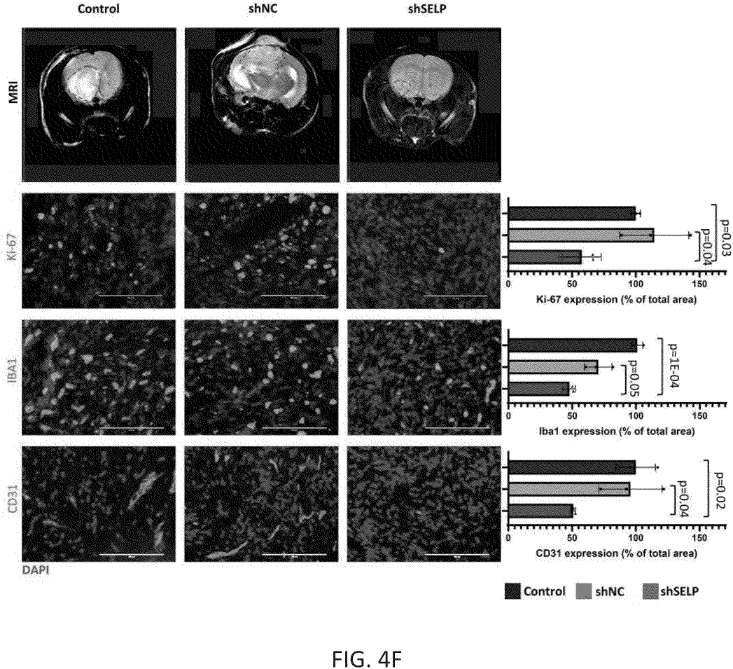

In order to evaluate the effect of SELP inhibition in vivo, we performed several experiments using murine and human GB mouse models. First, we evaluated the effect of SELP knockdown in human GB mouse models, we injected shSELP, shNC or control WT PD-GB4 or U251 cells, intracranially into SCID mice (FIGS. 4A-F). Mice bearing shSELP tumors exhibited delayed tumor growth and prolonged survival (FIGS. 4A, B, D, E). Ki-67 immunostaining showed reduced proliferation of GB cells in shSELP tumors and a low density of activated microglia and blood vessels in shSELP tumors was also observed by Iba1 and CD31 immunostaining, respectively (FIGS. 4C, F).

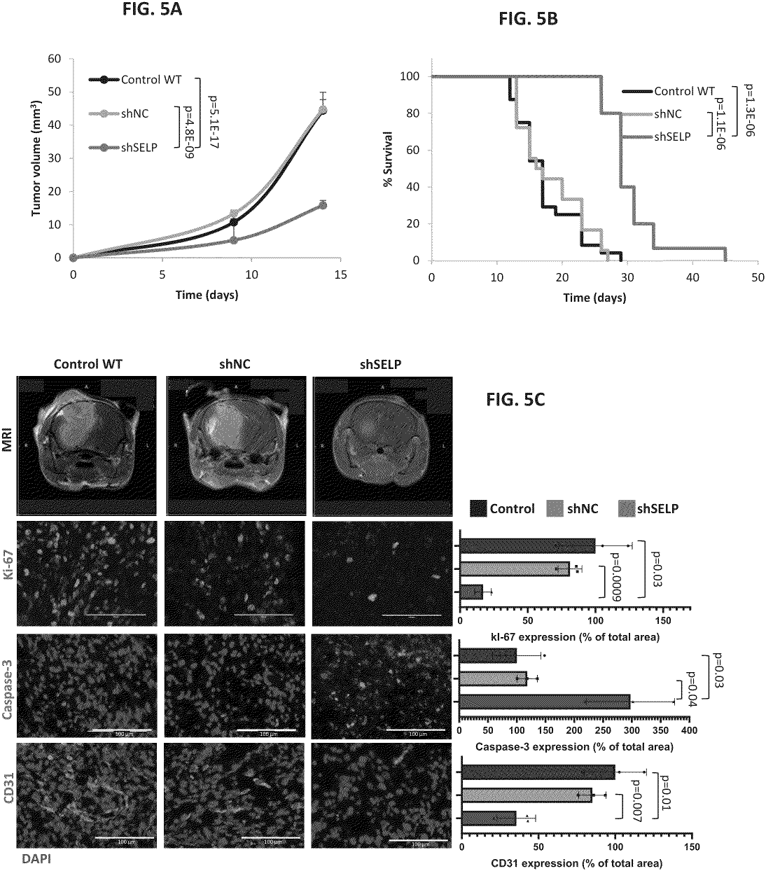

In order to investigate the effect of SELP knockdown on the adaptive immune response, we generated shSELP murine GL261 cells. Out of five different shRNA sequences, the shSELP plasmid which showed the highest silencing effect (approximately 90%) and delayed GL261 cell proliferation was chosen. Control WT, shSELP, and shNC GL261 cells, were injected intracranially into immunocompetent mice. Reduction in tumor growth and prolonged survival were observed in the shSELP group (FIGS. 5A, 5B). Flow cytometry and immunostaining analysis of the tumors revealed a higher percentage of CD8⁺ and lower percentage of CD4⁺/FOXP3⁺ T cells in shSELP tumors. In addition, immunostaining showed reduced proliferation (Ki-67), increased apoptosis (caspase-3), and a lower density of blood vessels (CD31) in shSELP tumors (FIG. 5C).

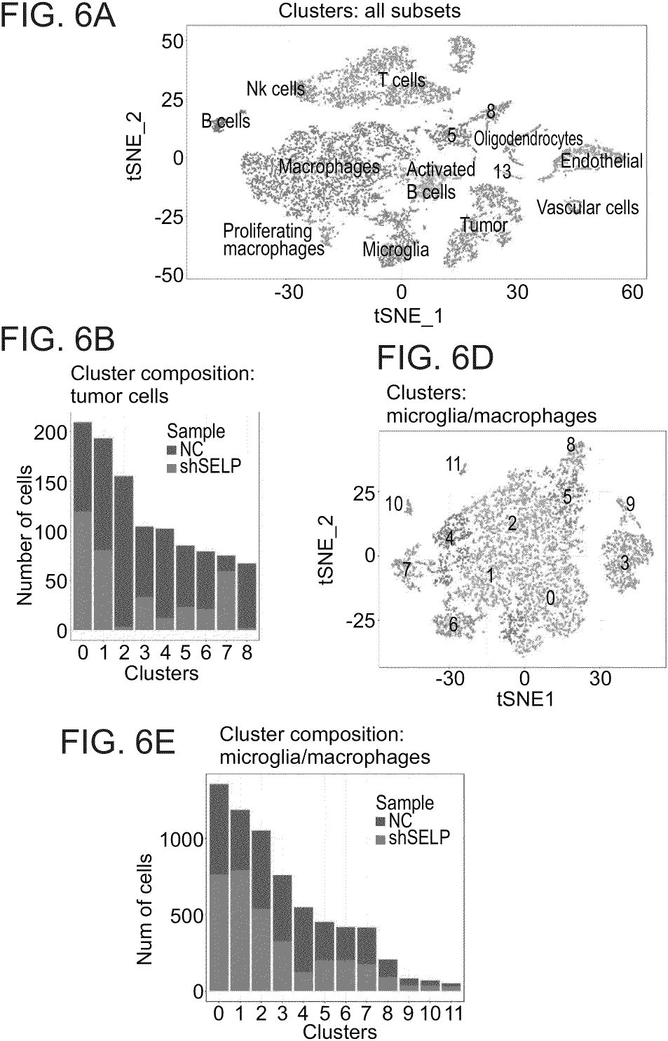

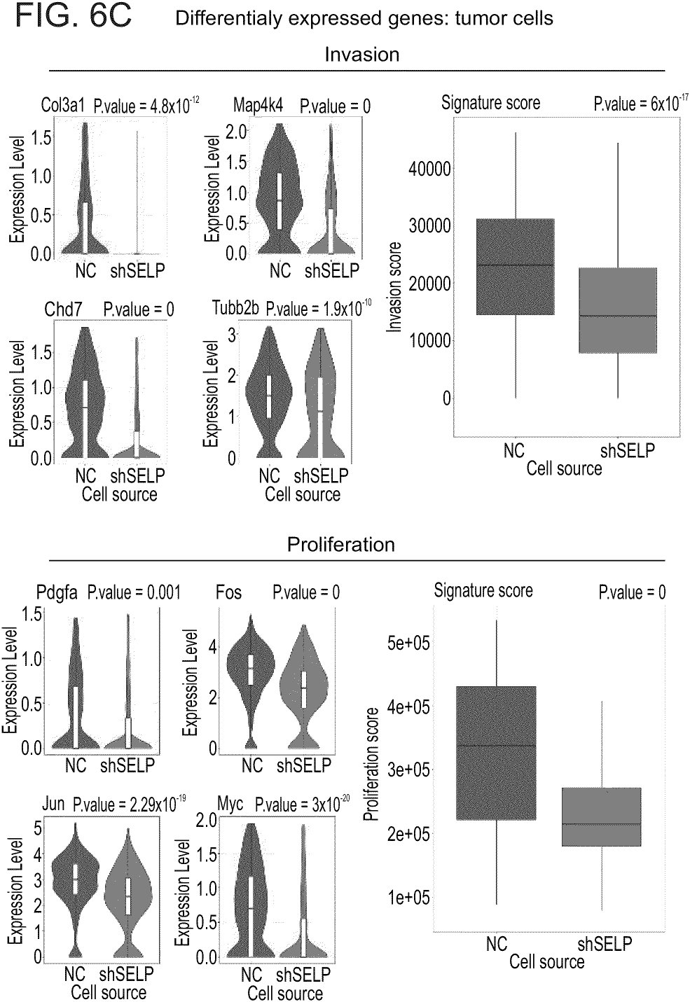

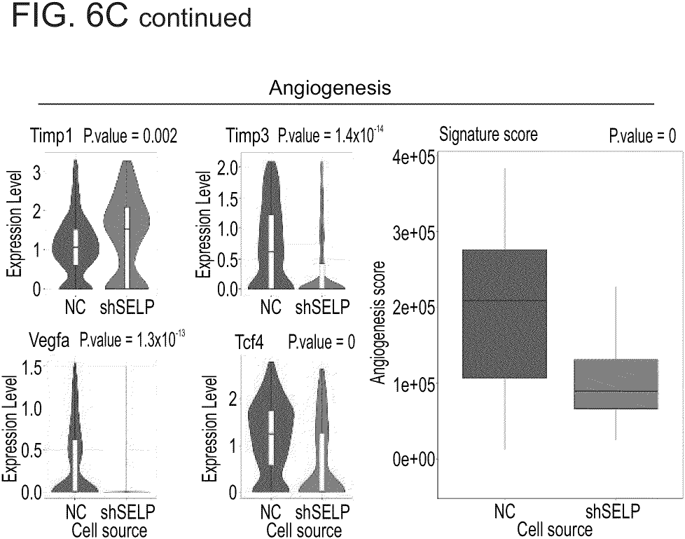

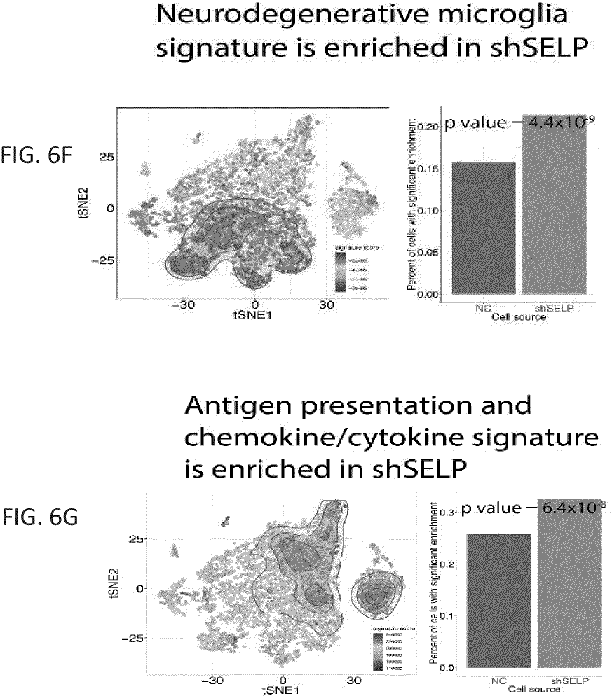

To further characterize the influence of SELP-PSGL-1 interactions on GB cells and the brain microenvironment, we performed single-cell RNA-seq of shNC and shSELP GL261 tumors focusing on three main subpopulations: tumor cells, T cells and microglia-macrophages (FIGS. 6A-I). We identified multiple clusters and were able to annotate most of them to known cell types with sufficient representation of our populations of interest (FIG. 6A). Deeper examination of the cancer-cell population revealed several tumor cell clusters that were differentially distributed between shNC and shSELP tumors (FIG. 6B, ). Within these clusters and following differential expression analysis we found genes related to cancer invasion such as Col3a1, Map4k4, Chd7 and Tubb2b [52-55], genes associated with proliferation and tumor progression such as Pdgfa and the oncogenes Fos, Jun and Myc [56-59], and angiogenesis genes as VEGFa, Tcf4, Timp1 and Timp3 [60-63] (FIG. 6C). Thus, we next applied unbiased, published signatures, representing these three processes on our data [64-66]. We found all three signatures related to cancer invasion, proliferation, and angiogenesis to be enriched in the shNC group (FIG. 6C). Within the brain microenvironment representing cell populations the microglia/macrophages clusters showed the most uneven internal distribution between shNC and shSELP cells. Thus, to get a better resolution of these changes, we further increased the number of cells analyzed by generating additional single-cell RNA-seq analysis using only CD11b positive cells (FIGS. 6D-G). Our focused single-cell RNA-seq data of freshly-isolated CD11b+ sorted microglia/macrophages from shSELP and shNC GL261 tumors, identified twelve sub-populations, termed clusters 0-11, that all expressed microglialmacrophages-related genes (FIGS. 6D-E). The three clusters (0, 1, and 4), which represent a large portion of the total population, appeared to have a different distribution of cells when the cells were isolated from SELP knockdown GB or control tumors. For example, clusters 0 and 1 had more cells in shSELP tumors and cluster 4 had more cells in control samples (FIG. 6E). Interestingly, GB-derived microglia/macrophages expressed a gene signature that strongly resembled the pro-inflammatory microglia signature (including genes such as Spp1, Il1b, and Cxcl9), described by Krasemann et al. in microglia derived from models of amyotrophic lateral sclerosis (ALS), multiple sclerosis (MS), and Alzheimer’s disease (AD). More importantly, perturbation of the SELP-PSGL-1 interactions, caused the microglia/macrophages cells to express an even higher score of this neurodegenerative signature (FIG. 6F). Another observation was that GB-derived microglia/macrophages expressed a variety of genes associated with antigen presentation, such as H2-Eb1, H2-Ab1, H2-Aa, and H2-DMb1, as well as immune cell recruiting factors such as Ccl2, Ccl3, Ccl4, Ccl5, Cxcl9, and Cxcl10. These were also enriched after manipulation of the SELP-PSGL-1 axis (FIG. 6G).

Signature enrichment algorithms demonstrated significant difference for both the neurodegenerative microglia signature score (p value = 2.2 × 10⁻¹⁶, Wilcoxon rank sum test and p value = 4.4 × 10⁻⁹ CERNO test, see Methods) and the antigen presentation and chemokine signature scores (p value = 8.969 × 10⁻¹⁶, Wilcoxon rank sum test andp value = 6.4 × 10⁻⁸ CERNO test, see Methods). Thus, as for T cells, it appears that common mechanisms are shared between microglia in autoimmunity-related inflammation and cancer immunity and this inflammatory environment can be further enhanced by perturbation of the SELP-PSGL-1 axis. In addition, we found reduced expression of PSGL-1 by microglia isolated from shSELP tumors compared to shNC showing the reciprocal effect of SELP knockdown in GB cells and its influence on the surrounding microglia. Of note, examination of the T cell representing clusters, showed similar cluster distribution between the shNC and shSELP groups. However, we did find several differentially expressed genes which represents T cell activation that were up-regulated in the shSELP group such as granzyme B, perforin, PD-1 and LAG-3.

The changes in antigen presentation and T cell recruitment signatures as well as the higher percentage of CD8⁺ T cell infiltration in the shSELP tumors and the minor differences found in the single-cell analysis, raise the question of the dependency of the observed in vivo effect on peripheral immunity.

However, our results using human GB mouse models show that the inhibitory effect of SELP knockdown is independent of T cells as these studies were performed using immunocompromised mice lacking the adaptive immune system. We note that caspase staining increased in shSELP tumors only in immunocompetent mice (FIG. 5C). This phenomenon could be correlated with enhanced infiltration of CD8+ T cells suggesting that these cells play a cytotoxic role in shSELP CB. Thus, the T cells may have partial contribution to the observed effects.

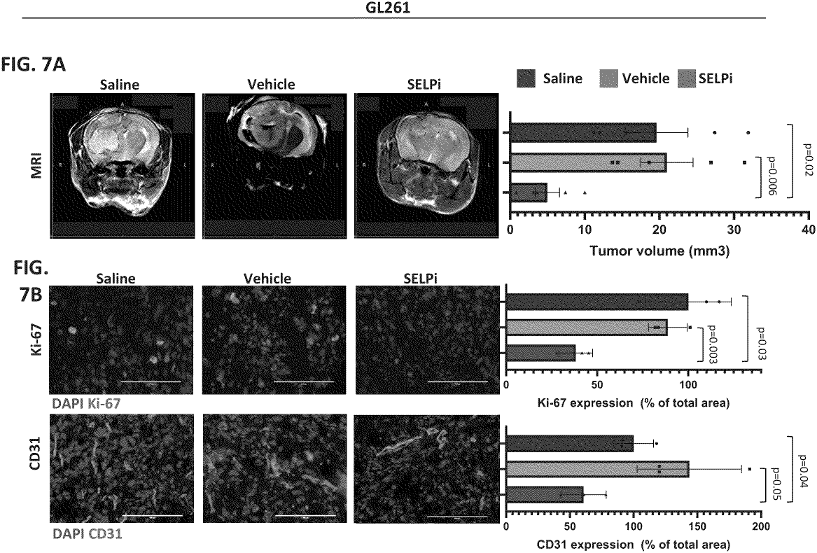

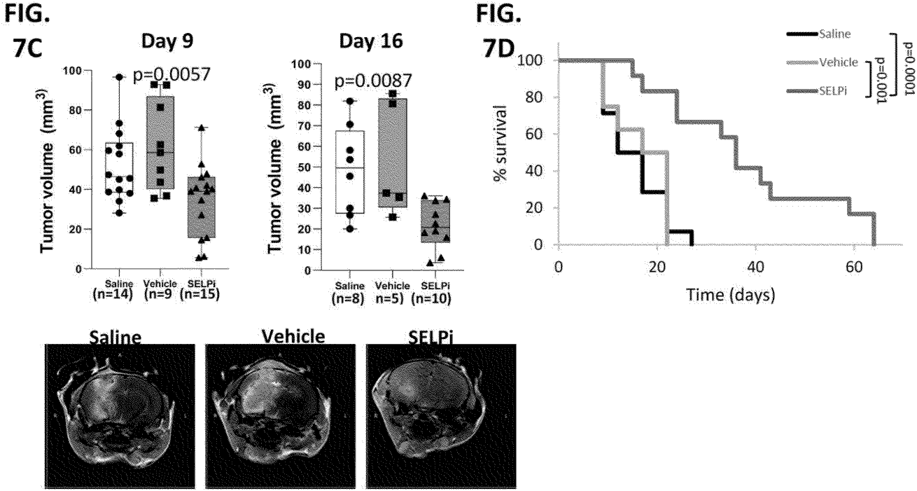

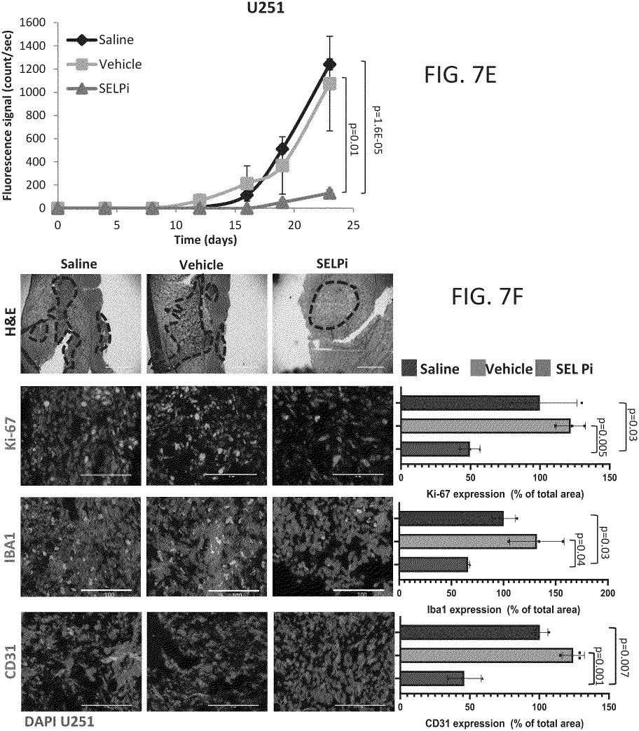

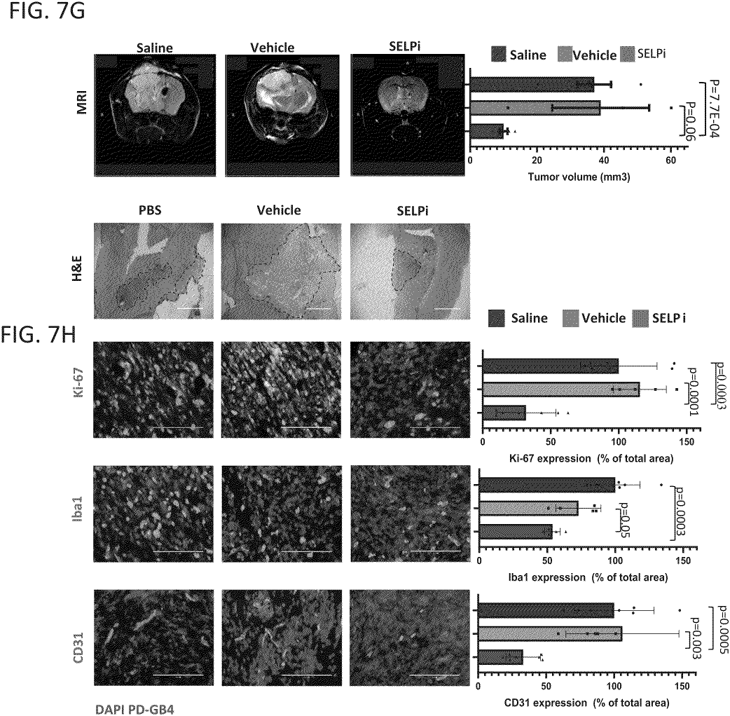

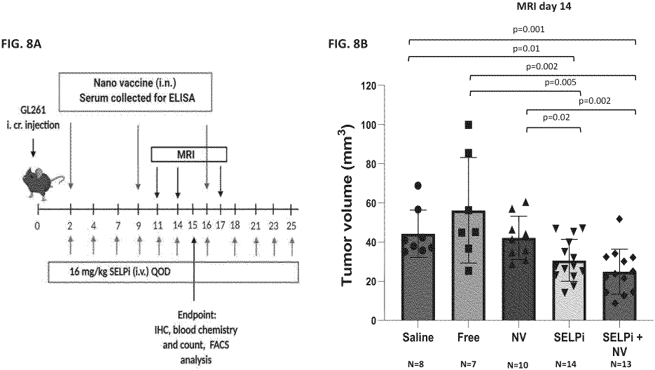

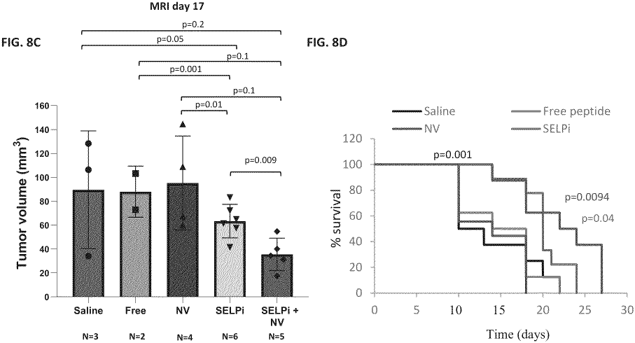

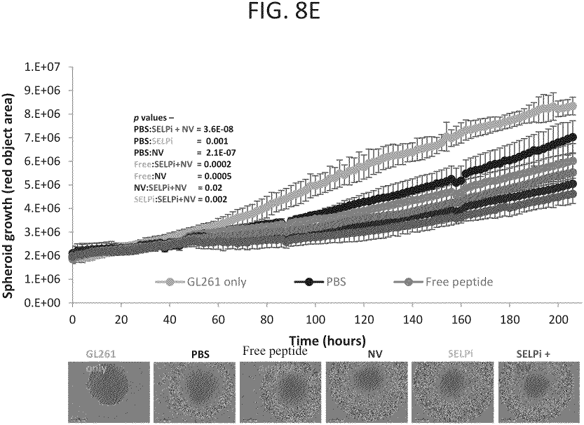

Finally, we evaluated the therapeutic potential of SELP inhibition for GB treatment. To this end, we treated GL261 and the lenti-induced, mesenchymal iAGR53 murine GB-bearing mice IV with SELPi. The results showed a significant reduction in tumor volume evaluated by MRI imaging in both models and prolonged survival for in the iAGR53 model (FIGS. 7A, C, D). In addition, immunostaining analysis of GL261 tumors displayed a clear reduction in proliferation and blood vessel density, increase of CD4+ and CD8+ T cells, and decrease in CD4+/FOXP3+ T cells in the tumors treated with SELPi (FIG. 7B. These results demonstrate the effect of SELP inhibition, not only on GB cells, but also on the brain microenvironment.

In our human GB mouse models, SELPi treatment of iRFP-labeled U251 cells, reduced the fluorescence signal detected by the CRI-Maestro™ imaging system (FIG. 7E). Immunostaining of these tumors revealed reduced invasion and proliferation of GB cells in the treated group, as shown by H&E and Ki-67 staining, respectively (FIG. 7F). In addition, we observed a reduction of blood vessel density (CD31) and microglia activation (Iba1) in the treated group (FIG. 7F).

In order to increase the signal obtained following IV treatment of SELPi, we used brain cannulas to inject SELPi intraventricularly into animals in the GB PDX mouse model (FIG. 7G). When we removed the cannulas at 21 days post injection, MRI imaging revealed a marked 67% reduction in tumor volume. We observed reduced invasion and proliferation of GB cells and reduction in Iba1 and CD31 density in SELPi treated PD-GB4 tumors (FIG. 7H).

As for validation that the observed effect on in vivo tumorigenesis is specifically due to SELP inhibition, we assessed for potential adverse effect of injecting SELPi systemically. Following single IV dose of 16 mg/kg injection of SELPi into healthy mice, blood chemistry results showed no significant changes between the saline, vehicle and SELPi groups. Complete blood count revealed an increase in the absolute amount of blood lymphocytes, which correlates with the results

What is claimed is:

1. A method of treating glioblastoma in a subject in need thereof comprising administering to the subject a therapeutically effective amount of an agent that specifically decreases an amount and/or activity of P-selectin, thereby treating the glioblastoma.

2. The method of claim 1, wherein said agent specifically binds to P-selectin or a polynucleotide encoding said P-selectin.

3. The method of claim 1, wherein said agent binds to P-Selectin glycoprotein ligand-1 (PSGL-1) or a polynucleotide encoding said PSGL-1.

4. The method of claim 1, further comprising administering to the subject an immunomodulatory agent.

5. The method of claim 4, wherein said immunomodulatory agent comprises an immunomodulatory antibody.

6. The method of claim 5, wherein said immunomodulatory antibody is selected from the group consisting of anti-CTLA4, anti-CD40, anti-41BB, anti-OX40, anti-PD1, anti-PDL1, anti-LAG3, anti-IDO, and anti-TIGIT.

7. The method of claim 1, wherein said agent is an inhibitory antibody that binds to and inhibits said P-selectin.

8. The method of claim 7, wherein said inhibitory antibody is attached to a therapeutic agent.

9. The method of claim 7, wherein said inhibitory antibody is not attached to a therapeutic agent.

10. The method of claim 1, wherein said agent is a small molecule agent.

11. The method of claim 1, wherein said agent is a polynucleotide agent.

12. The method of claim 4, wherein said agent is co-formulated with said immunomodulatory agent.

13. The method of claim 1, wherein said agent is administered following resection of said glioblastoma tumor.

14. The method of claim 1, wherein said glioblastoma is an early stage glioblastoma.

15. The method of claim 1, wherein said agent is comprised in a nanoparticle.

16. The method of claim 15, wherein said nanoparticle is attached to a targeting moiety that increases delivery across the blood brain barrier.

17. The method of claim 1, wherein said agent is attached to a targeting moiety that increases delivery across the blood brain barrier.

18. A pharmaceutical composition comprising a pharmaceutically acceptable carrier and:
(i) an active agent that specifically decreases an amount and/or activity of P-selectin; and
(ii) an immunomodulatory agent, as a second active agent.

19. An article of manufacture comprising:
(i) an active agent that specifically decreases an amount and/or activity of P-selectin; and
(ii) an immunomodulatory agent.


SEQUENCE LISTING (18 sequences)

SEQ ID NO: 1 - DNA, 22 bp, synthetic construct
  misc_feature 1..22: note = Single strand DNA oligonucleotide
  source 1..22: mol_type = other DNA
tccttgagagcgtttcagtatg

SEQ ID NO: 2 - DNA, 22 bp, synthetic construct
  misc_feature 1..22: note = Single strand DNA oligonucleotide
  source 1..22: mol_type = other DNA
ctgtccactgtcccaagttatc

SEQ ID NO: 3 - DNA, 22 bp, synthetic construct
  misc_feature 1..22: note = Single strand DNA oligonucleotide
  source 1..22: mol_type = other DNA
gactttgagctactgggatctg

SEQ ID NO: 4 - DNA, 23 bp, synthetic construct
  misc_feature 1..23: note = Single strand DNA oligonucleotide
  source 1..23: mol_type = other DNA
caggaagtgatgttatgcctttg

SEQ ID NO: 5 - DNA, 18 bp, synthetic construct
  misc_feature 1..18: note = Single strand DNA oligonucleotide
  source 1..18: mol_type = other DNA
cgcatgtgaactccctgg

SEQ ID NO: 6 - DNA, 21 bp, synthetic construct
  misc_feature 1..21: note = Single strand DNA oligonucleotide
  source 1..21: mol_type = other DNA
tagatgcctttctcttggagc

SEQ ID NO: 7 - DNA, 20 bp, synthetic construct
  misc_feature 1..20: note = Single strand DNA oligonucleotide
  source 1..20: mol_type = other DNA
gcctttcctgcttctcatgg

SEQ ID NO: 8 - DNA, 21 bp, synthetic construct
  misc_feature 1..21: note = Single strand DNA oligonucleotide
  source 1..21: mol_type = other DNA
gtacattgacttccgcaagga

SEQ ID NO: 9 - DNA, 20 bp, synthetic construct
  misc_feature 1..20: note = Single strand DNA oligonucleotide
  source 1..20: mol_type = other DNA
attccacccatggcaaattc

SEQ ID NO: 10 - DNA, 21 bp, synthetic construct
  misc_feature 1..21: note = Single strand DNA oligonucleotide
  source 1..21: mol_type = other DNA
ggatctcgctcctggaagatg

SEQ ID NO: 11 - DNA, 20 bp, synthetic construct
  misc_feature 1..20: note = Single strand DNA oligonucleotide
  source 1..20: mol_type = other DNA
cgcctttgctcatgacattg

SEQ ID NO: 12 - DNA, 20 bp, synthetic construct
  misc_feature 1..20: note = Single strand DNA oligonucleotide
  source 1..20: mol_type = other DNA
tcaaacgtctcacaggctgc

SEQ ID NO: 13 - DNA, 20 bp, synthetic construct
  misc_feature 1..20: note = Single strand DNA oligonucleotide
  source 1..20: mol_type = other DNA
aggtctgtgggaaaagcaag

SEQ ID NO: 14 - DNA, 17 bp, synthetic construct
  misc_feature 1..17: note = Single strand DNA oligonucleotide
  source 1..17: mol_type = other DNA
gccagagattccaattg

SEQ ID NO: 15 - DNA, 22 bp, synthetic construct
  misc_feature 1..22: note = Single strand DNA oligonucleotide
  source 1..22: mol_type = other DNA
caccttggtcttggagcttatt

SEQ ID NO: 16 - DNA, 19 bp, synthetic construct
  misc_feature 1..19: note = Single strand DNA oligonucleotide
  source 1..19: mol_type = other DNA
ggggctgatcccgttgatt

SEQ ID NO: 17 - DNA, 21 bp, synthetic construct
  misc_feature 1..21: note = Single strand DNA oligonucleotide
  source 1..21: mol_type = other DNA
tgaattccctgggtgagaagc

SEQ ID NO: 18 - DNA, 20 bp, synthetic construct
  misc_feature 1..20: note = Single strand DNA oligonucleotide
  source 1..20: mol_type = other DNA
actggagttgtacggcagtg